SEQUENCE LISTING (17 sequences)

SEQ ID NO: 1 - DNA, 21 bp, Artificial Sequence
  Description of Artificial Sequence Synthetic primer
ctgggtagtttgtaagcatgc

SEQ ID NO: 2 - DNA, 21 bp, Artificial Sequence
  Description of Artificial Sequence Synthetic primer
caataaactgctggtctcagg

SEQ ID NO: 3 - DNA, 30 bp, Artificial Sequence
  Description of Artificial Sequence Synthetic primer
atcgccttctatcgccttcttgacgagttc

SEQ ID NO: 4 - DNA, 22 bp, Artificial Sequence
  Description of Artificial Sequence Synthetic primer
agttgtgatcgtgcagccaagc

SEQ ID NO: 5 - DNA, 22 bp, Artificial Sequence
  Description of Artificial Sequence Synthetic primer
acaagacgtgccttgcacagct

SEQ ID NO: 6 - DNA, 23 bp, Artificial Sequence
  Description of Artificial Sequence Synthetic primer
tgataagttgtcttggggtttcc

SEQ ID NO: 7 - DNA, 19 bp, Artificial Sequence
  Description of Artificial Sequence Synthetic primer
ctgtgtctgacgcacctgt

SEQ ID NO: 8 - DNA, 20 bp, Artificial Sequence
  Description of Artificial Sequence Synthetic primer
ggctggtttactgtgtcttt

SEQ ID NO: 9 - DNA, 20 bp, Artificial Sequence
  Description of Artificial Sequence Synthetic primer
tggaaatactcctgatgcag

SEQ ID NO: 10 - DNA, 22 bp, Artificial Sequence
  Description of Artificial Sequence Synthetic primer
tgggtggacaggtgaacttgct

SEQ ID NO: 11 - DNA, 22 bp, Artificial Sequence
  Description of Artificial Sequence Synthetic primer
acccaagctttccatcctggga

SEQ ID NO: 12 - DNA, 22 bp, Artificial Sequence
  Description of Artificial Sequence Synthetic primer
tctggctcacgcttccacaaca

SEQ ID NO: 13 - DNA, 22 bp, Artificial Sequence
  Description of Artificial Sequence Synthetic primer
ttttcaggcatctcggggctgt

SEQ ID NO: 14 - DNA, 23 bp, Artificial Sequence
  Description of Artificial Sequence Synthetic primer
agaaacatggaattaacgtggct

SEQ ID NO: 15 - DNA, 23 bp, Artificial Sequence
  Description of Artificial Sequence Synthetic primer
aaatgcagtcaagaatcctggtt

SEQ ID NO: 16 - DNA, 20 bp, Artificial Sequence
  Description of Artificial Sequence Synthetic primer
ctggagaaacctgccaagta

SEQ ID NO: 17 - DNA, 20 bp, Artificial Sequence
  Description of Artificial Sequence Synthetic primer
tgttgctgtagccgtattca

(19) United States
(12) Patent Application Publication
(10) Pub. No.: US 2019/0183910 A1
(43) Pub. Date: Jun. 20, 2019

(54) ENHANCING DNA REPAIR AND PREVENTING AGING AND ITS CONSEQUENCES IN REPRODUCTIVE AND OTHER CELLS

(71) Applicant: NEW YORK MEDICAL COLLEGE, Valhalla, NY (US)

(72) Inventor: KUTLUK OKTAY, RYE, NY (US)

(73) Assignee: KUTLUK OKTAY, RYE, NY (US)

(21) Appl. No.: 16/311,969

(22) PCT Filed: Jun. 21, 2017
(86) PCT No.: PCT/US17/38456

§ 371 Date: Dec. 20, 2018

Related U.S. Application Data
(60) Provisional application No. 62/353,883, filed on Jun. 23, 2016.

(51) Int. Cl. A61K 31/661; A61P 15/08; C12N 5/076

(52) U.S. Cl. CPC A61K 31/661; C12N 5/061; A61P 15/08

(57) ABSTRACT

Embodiments of the present invention generally relate to methods for improving male fertility, and in particular for using sphingosine 1 phosphate (S1P) for improving the quality and/or longevity of sperm.

### CROSS REFERENCE TO RELATED APPLICATIONS

The present application claims priority to provisional application U.S. Patent Application No. 62/353,883 filed Jun. 23, 2016, which is incorporated by reference in its entirety.

### SEQUENCE LISTING

The instant application contains a Sequence Listing which has been filed electronically in ASCII format and is hereby incorporated by reference in its entirety. Said ASCII copy, created on Jun. 16, 2017, is named 005174-WO0_SL.txt and is 4,141 bytes in size.

### FIELD OF THE INVENTION

Embodiments of the present invention generally relate to methods for improving male fertility, and in particular for using sphingosine 1 phosphate (S1P) for improving the quality and/or longevity of sperm. Moreover, embodiments relate to enhancing DNA repair and reversing reproductive and general aging.

### BACKGROUND

There is mounting evidence on the impact of paternal age on genetic integrity. Since 1980, the age of paternity for men in their 30's and 40's has increased by 21% and nearly 30% respectively (Harris et al., 2011). A male partner age of greater than 40 years contributes to reduced fertility and fecundity of a couple, especially when the female partner is also of advanced age (Kuhnert and Nieschlag, 2004). With increased age in men, sperm may become genetically defective and their quality may decrease, which increases the chances of germ line mutations (Fisch, 2009). Considering that men and women are postponing having children until later in life, aging emerges as a detrimental factor on the quality of life and health of the offspring.

Paternal age has been suggested to affect sperm genetics by its association with chromosomal abnormalities, mutations, and DNA damage (Chianese et al., 2014). Sources of DNA damage that affect male and female fertility include damage from oxidative stress, UV exposure, posttranslational modification errors, and damage from cancer with chemotherapy agents. Spermatozoa membranes are highly-susceptible to oxidative damage due to the peroxidation of lipid membranes, damaging the sperm and rendering them incapable of fertilizing the ovum (Harris et al., 2011). Although reactive oxygen species are necessary for the physiological functions of sperm including capacitation and hyper-activation, an imbalance between production and removal of ROS leads to oxidative stress, which is a main factor in male infertility (Bejarano et al., 2014).

There are presently no treatments to address sperm aging or damage. There is an ongoing need for methods and treatments for stabilizing and counteracting age-related damage to sperm.

Furthermore, DNA repair declines with age in human oocytes (Titus et al., 2013) and that mechanism of oocyte aging is due to this age related decline in DNA repair. Moreover, insufficient DNA repair is associated with many cancers and other lethal conditions. Hence enhancing DNA repair may not only slow down sperm and egg aging but it may also reduce cancer risks and reverse aging in general. It is therefore important to enhance DNA repair so as to slow down or reverse reproductive and general aging.

### DESCRIPTION OF THE DRAWINGS

FIGS. 1A-D are graphs showing the improved quality in S1P-treated mouse sperm.

FIGS. 2A-F are graphs showing increased expression of DNA repair genes in S1P-treated mouse sperm.

FIGS. 3A-C are graphs showing sperm quality parameters in S1P-treated mouse sperm with an ATM inhibitor, which confirms that the effect of S1P is through enhancing DNA repair in the ATM-mediated repair pathway.

FIGS. 4A-F are graphs illustrating decreased expression of DNA repair genes in S1P-treated mouse sperm with ATM inhibitor, which confirms that the effect of S1P is through enhancing DNA repair in the ATM-mediated repair pathway.

FIGS. 5A-H are graphs showing the results of alkaline and neutral comet assay analysis on mouse and human sperm.

### SUMMARY OF THE INVENTION

Disclosed herein is a composition comprising live sperm, and at least one stabilizing agent effective in increasing the lifespan or stability of the sperm, and optionally a physiologically acceptable carrier.

In one embodiment, the stabilizing agent comprises an effective amount sphingosine 1 phosphate (S1P), a pharmaceutically acceptable salt thereof, or an analog thereof. In one embodiment, the composition further comprises at least one cryoprotectant.

The disclosure also provides a kit of the composition and optionally a device for self-administration.

Provided herein is a method of treating male reproductive system by administering a composition comprising sphingosine-1-phosphate, a pharmaceutically acceptable salt thereof, or an analog thereof, in an amount sufficient to enhance DNA repair to protect against a natural or artificial insult, wherein the administration is in vivo or ex vivo. In one embodiment, the natural insult is aging and the artificial insult is chemotherapeutic drugs or radiation treatment.

Provided herein is a method to protect a male or a female subject against overall aging, reverse the effects of aging and to extend lifespan by administering to the subject a composition comprising sphingosine-1-phosphate, a pharmaceutically acceptable salt thereof, or an analog thereof in an effective amount sufficient to enhance DNA repair, wherein the administration is in vivo or ex vivo.

Provided herein is a method for improving functionality and/or fertility of sperm, comprising contacting the sperm with an effective amount of sphingosine 1 phosphate (S1P), a pharmaceutically acceptable salt thereof, or an analog thereof.

In certain embodiments, the S1P analog is N,N-dimethylsphingosine-1-phosphate; N,N,N-trimethylsphingosine-1-phosphate; N-acetylsphingosine-1-phosphate; N-acylsphingosine-1-phosphate; sphingosine-1,3-diphosphate; sphingosine-3-phosphate; sphingosine-1-thiophosphate; N,N-dimethylsphingosine-1-thiophosphate; N,N,N-trimethylsphingosine-1-thiophosphate; or pharmaceutically acceptable salts thereof.

In certain embodiments, the effective amount of S1P, pharmaceutical salt thereof or analog thereof is 0.001 milligram to 200 milligrams per kilogram body weight.

Disclosed herein is a method for improving an outcome in artificial insemination which comprises contacting an oocyte with an effective amount of the composition. In certain embodiments, the artificial insemination includes intrauterine, intracervical, or intravaginal routes, and assisted reproductive techniques include gamete intrafallopian transfer (“GIFT”), zygote intrafallopian transfer, ICSI, and in vitro fertilization followed by embryo cryopreservation or transfer.

In certain embodiments, the method further comprises the steps of:

(i) retrieving an oocyte via a transvaginal oocyte retrieval with or without prior controlled ovarian stimulation;
(ii) retrieving sperms via masturbation, ejaculation, electro-ejaculation, prostate massage, centrifugation of urine post ejaculation in case of retrograde ejaculation, TESE, TESA, micro-TESE, from fresh testicular tissues, frozen testicular tissues, freshly removed testis or frozen testis;
(iii) treating the sperms, testis or testicular tissues with S1P, a pharmaceutically acceptable salt thereof or an analog thereof from 1 min to 3 days in vitro prior to artificial insemination, in vitro fertilization, intracytoplasmic sperm injection, GIFT or ZIFT;
(iv) combining the sperms and oocyte in the presence of S1P to form an embryo; and
(v) transferring the embryo into the fallopian tube or uterine cavity of a subject via laparoscope, hysteroscope, or optionally, cryopreserving the embryo.

Also disclosed is a method for cryopreserving sperm comprising: (a) contacting the composition disclosed herein with a cryoprotectant; and (b) storing the composition at a temperature of about 4° C. to about −196° C.

Disclosed herein is a method of protecting the reproductive system of a male subject against a chemical or radiation insult, comprising: administering in vivo or ex vivo to said male subject a composition comprising an agent that antagonizes one or more acid sphingomyelinase (ASMase) gene products and/or enhances DNA repair, wherein said agent is a lysophospholipid, in an amount sufficient to protect the reproductive system of said male subject from pre-mature aging or destruction caused by said chemical or radiation insult.

In certain embodiments, the chemical insult includes cytotoxic factors, chemotherapeutic drugs, hormone deprivation, growth factor deprivation, cytokine deprivation, cell receptor antibodies, or a combination thereof.

In certain embodiments, the chemotherapeutic drug includes cyclophosphamide, melphalan, any other member of the alkylating agents category of chemotherapeutics, doxorubicin or any other member of the topoisomerase inhibitor category of chemotherapeutic drugs, 5FU, methotrexate, or any other member of the antimetabolite category chemotherapeutic drugs, vinblastine, actinomycin D, etoposide, cisplatin, taxotere, taxol or a combination thereof.

In certain embodiments, the radiation insult includes ionization radiation, x-ray, infrared radiation, ultrasound radiation, heat, or a combination thereof.

In certain embodiments, the radiation insult includes an invasive radiation therapy, a non-invasive radiation therapy, or a combination thereof.

In one embodiment, the male reproductive system comprises testes. In one embodiment, the male reproductive system comprises sperms. In one embodiment, the male is in a reproductive age. In one embodiment, the male is in a pre-reproductive age. In one embodiment, the male is in a post-reproductive age.

In one embodiment, the lysophospholipid is a sphingolipid compound, pharmaceutical salt thereof or an analog thereof. In one embodiment, the sphingolipid compound is S1P, a pharmaceutical salt thereof or an analog thereof.

In one embodiment, the composition is administered at least once from about fifteen days to about two days prior to exposure to said insult. In one embodiment, the composition is administered at about seven days to about two hours prior to exposure to said insult. In one embodiment, the composition is administered regularly for a continuous period of time. In certain embodiments, the composition is administered orally, intravascularly, intraperitoneally, subcutaneously, intramuscularly, inter-uterine, intra-ovarian, intratesticular, inta-urethral, rectally, topically, or a combination thereof.

In certain embodiments, the chemical or radiation insult is a result of a therapy against a disease or a disorder. In certain embodiments, the disease or disorder comprises cancer, rheumatoid arthritis, angioplasy, or restenosis. In certain embodiments, the cancer comprises colon carcinoma, pancreatic cancer, breast cancer, ovarian cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acute lymphocytic leukemia and acute myelocytic leukemia, chronic leukemia and polycythemia vera, lymphoma (Hodgkin's disease and non-Hodgkin's disease), multiple myeloma, Waldenstrom's macroglobulinemia, or a combination thereof.

In one embodiment, the administration is an ex vivo administration. In one embodiment, the administration is an in vivo administration. In one embodiment, the male subject is exposed to a chemical insult prior to the radiation insult. In one embodiment, the male subject is exposed to a radiation insult prior to the chemical insult. In one embodiment, administration of the composition is terminated prior to, concurrently with or subsequent to the chemical insult. In certain embodiments, administration of said composition is terminated prior to, concurrently with or subsequent to said radiation insult.

Disclosed herein is a method to enhance DNA repair or reduce cancer risk of a subject comprising administering an effective amount of acid sphingomyelinase (ASMase) antagonist. In one embodiment, the antagonist is lysophospholipid. In one embodiment, the lysophospholipid is a sphingolipid compound, pharmaceutical salt thereof or an analog thereof.

In one embodiment, the sphingolipid compound is S1P, a pharmaceutical salt thereof or an analog thereof.

### DETAILED DESCRIPTION

Sphingosine-1-phosphate (S1P) is a sphingolipid metabolite which regulates many processes in a cell, such as survival and proliferation (Guo et al., 2014). S1P acts in triggering diverse cellular responses that include proliferation, migration, and cytoskeletal changes. S1P is an anti-apoptotic agent and is present abundantly in the blood. The way S1P functions as an anti-apoptotic agent is possibly by suppressing ceramide, which is an intracellular mediator of apoptosis (Hannun, 1996).

Several studies have shown that S1P protects somatic cells (Ishii et al., 2009) (Aoki et al., 2016). S1P has been shown to block ceramide induced apoptosis in oocytes ex vivo and prevent developmental apoptosis and apoptosis induced by anti-cancer therapy in oocytes (Morita et al., 2000) (Tilly and Kolesnick, 2007). Ceramide-induced death pathway has been shown to be involved in radiation therapy and chemotherapy-induced oocyte death (Beckham et al., 2013) (Li et al., 2014). As per Suomalainen, et al, ceramide levels were found to increase rapidly before appearance of caspase 3 activation and DNA laddering in male germ cell apoptosis; suggesting that there is a role for ceramide in the induction of germ cell death (Suomalainen et al., 2003). They also found that ceramide appeared to regulate an early step of apoptosis because its levels were unchanged when a downstream part of the pathway was blocked. Male germ cell loss by radiation-induced injury has been shown to be decreased with S1P treatment to mouse testes (Otala et al., 2004). However, how S1P prevented germ cells against such genotoxic injury has not been shown, until the data presented in this application.

The present invention relates to therapeutic and/or stabilized sperm compositions or formulations as described herein. The data illustrate that S1P treatment increases sperm quality, and/or diminishes or reverses aging of sperm by enhancing DNA repair, and provides for stable sperm compositions that can be used for present or future fertility treatments.

Embodiments of the present invention also relate to a method of protecting the reproductive system of a male subject against a chemical or radiation insult, comprising: administering in vivo or ex vivo to said male subject a protective composition comprising an agent that antagonizes one or more acid sphingomyelinase (ASMase) gene products, wherein said agent is a lysophospholipid, sphingolipid compound, and more specifically sphingosine I-phosphate, in an amount sufficient to protect the reproductive system of said male subject from pre-mature aging or destruction caused by said chemical or radiation insult. Other agents in this category include but not limited to various S1P analogs and S1P receptor agonists such as A-971432 (Hobson et al., 2015), FTY720 and SEW2871 (Soleimani et al., 2011).

In one embodiment, disclosed herein is a method of treatment comprising administering S1P, a pharmaceutically acceptable salt thereof, or an analog thereof via one or more of the following route: oral, transdermal, intramuscular, subcutaneous, rectal, nasal or other routes that are known in the art, rendering systemic absorption. In an embodiment, S1P, a pharmaceutically acceptable salt thereof, or an analog thereof is administered via direct injection to testis. The S1P, a pharmaceutically acceptable salt thereof, or an analog thereof is administered in an effective amount and for a sufficient period of time. A sufficient period of time is determined by one skilled in the art which can range from 1 day to life time to retard or prevent sperm aging, enhance sperm quality or prevent sperm from exposure to chemotherapy agents or radiation treatment to testis or lower abdominal area. In certain embodiments, the duration of treatment is 1-5 days, 5-10 days, 10-20 days, 20-30 days, 1-2 months, 2-4 months, 4-6 months, 6-12 months, 1-3 years, 3-6 years, 6-10 years, 10-20 years, 20-30 years, 30-40 years, 40-60 years. In certain embodiments, chemotherapeutic agents include but not limited to alkylating agents, topoisomerase inhibitors, antimetabolites, cyclophosphamide, melphalan, doxorubicin, 5FU, methotrexate, vinblastine, actinomycin D, etoposide, cisplatin, taxotere, taxol or a combination thereof. In certain embodiments, the composition described herein is administered on a continuous, periodic, or temporary basis. Depending on the type of insult and objectives of the therapy intended, for example, if protection of the male reproductive system from long term insults such as aging is intended, administration of the composition disclosed herein on a continuous or periodic basis is preferred. In a continuous administration, the composition is generally administered regularly, on a predetermined interval, for an indefinite period of time. Predetermined intervals comprise daily, weekly, biweekly, monthly, or yearly intervals depending on the type of S1P compound and the context.

Likewise, because S1P, a pharmaceutically acceptable salt thereof, or an analog thereof enhances DNA repair and declining DNA repair is a key mechanism in overall aging, in certain embodiments, the composition disclosed herein is administered to a subject to slow down or reverse overall aging of the subject. In one embodiment, the subject is human. In one embodiment, the subject is male. In one embodiment, the subject is female. In certain embodiments, the subject is an animal. In certain embodiments, the subject is a livestock or endangered animals. In certain embodiments, the composition disclosed herein is administered in a continuous or periodic basis. In certain embodiments, the composition is administered regularly, on a predetermined interval, for an indefinite period of time. In certain embodiments, predetermined intervals comprise daily, weekly, biweekly, monthly, or yearly intervals depending on the type of S1P compound and the context.

In certain embodiments, the method disclosed herein is used for the prevention of chemotherapy-induced or radiation therapy-induced damage to sperm. In certain embodiments, the treatment is initiated 1 day to 1 month before the chemotherapy or radiation therapy and continued throughout the course of the chemotherapy or radiation therapy. In certain embodiments, the treatment is terminated 1-5, 5-10, 10-15, 15-20, 20-30 days after the last administration of the chemotherapy or radiation therapy. In certain embodiment, the chemotherapy includes the administration of alkylating agents, topoisomerase inhibitors, antimetabolites, cyclophosphamide, melphalan, doxorubicin, 5FU, methotrexate, vinblastine, actinomycin D, etoposide, cisplatin, taxotere, taxol or a combination thereof. In case of testicular injection, the method is accomplished by percutaneous needle injection in the epididymis or seminiferous tubules. In the case of chemotherapy-induced or radiotherapy-induced damage to sperm, in one embodiment, the composition described herein is administered via continuous infusion, continuous transdermal delivery or continuous or intermittent nasal delivery.

The dosage of the therapeutic agent is adjusted according to, for example, the duration and the objective of the treatment intended. In one embodiment, a lower dosage maybe required for long term administration.

Embodiments of the present invention include a composition comprising live sperm, and at least one stabilizing agent effective in increasing the lifespan of the sperm, and optionally a physiologically acceptable carrier. Additional embodiments include compositions wherein the stabilizing agent comprises an effective amount sphingosine 1 phosphate (S1P), a pharmaceutically acceptable salt thereof, or an analog thereof. Additional embodiments include compositions further comprising at least one cryoprotectant. Suitable cryoprotectans include, glycerol, propanediol, dimethysulphoxide (DMSO), ethylene glycol or trehalose.

Embodiments of the present invention also relate to a method for improving functionality and/or fertility of sperm, comprising contacting the sperm with an effective amount of sphingosine 1 phosphate (S1P), a pharmaceutically acceptable salt thereof, or an analog thereof. In one embodiment, the method is used to treat a subject with low sperm motility, high percentage of abnormally shaped sperm or sperms that fertilize eggs resulting in pregnancies with poor embryo development, recurrent miscarriages, birth defects or diseases in offspring that are associated with poor sperm quality or male aging. In certain embodiments, S1P, a pharmaceutically acceptable salt thereof, or an analog thereof is administered systemically or locally. In certain embodiments, the administration is continuous or intermittent. In certain embodiments, the administration is in vivo or in vitro. In certain embodiments, systemic administration begins 1-10 days, 10-20 days, 20-30 days, 1-2 months, 2-4 months, 4-6 months, 6-8 months, 8-10 months, 10-12 months before the attempt of pregnancy albeit naturally or with assisted reproduction methods including intrauterine insemination, intracervical insemination, intravaginal insemination, gamete intrafallopian transfer (GIFT), zygote intrafallopian transfer (ZIFT), or in vitro fertilization, until a pregnancy is achieved. In the case of in vitro administration, sperm can be cultured with S1P, a pharmaceutically acceptable salt thereof, or an analog thereof from 1-10 mins, 10-30 mins, 30-60 mins, 1-2 hours, 2-5 hours, 5-10 hours, 10-24 hours, 1-2 days, or 2-3 days before the sperm is used for insemination, GIFT, ZIFT, or in vitro fertilization.

Embodiments of the present invention also relate to a method for preparing a composition for use in artificial insemination or in vitro fertilization comprising: contacting an egg with an effective amount of a composition comprising live sperm, and at least one stabilizing agent effective in increasing the lifespan or stability of the sperm, and optionally a physiologically acceptable carrier.

Embodiments of the present disclosure also relate to a method for cryopreserving sperm comprising: (a) contacting the composition of claim 1 with a cryoprotectant and (b) storing the composition at a temperature of about 4° C. to about −196° C. To enhance the health of sperm in cryo storage, sperm will be collected via masturbation, electroejaculation, needle aspiration (testicular sperm aspiration-TESA, testicular sperm extraction-TESE, microscopic testicular sperm extraction-microTESE, testicular tissue biopsy). Sperm will first be incubated with S1P from 1-10 mins, 10-30 mins, 30-60 mins, 1-2 hours, 2-5 hours, 5-10 hours, 10-24 hours, 1-2 days, or 2-3 days. It will then be mixed with a composition containing S1P or on of its derivatives and a cryoprotectant and will be frozen by vitrifcation or slow freezing methods.

Age plays a key role in the reproductive potential of both sexes. In females, fertility and oocyte quality decline with age, essentially precluding livebirths after ages 45-46 and resulting in significant increase in chromosomal abnormalities in the preceding decade (Battaglia et al., 1996). BRCA-related DNA DSB repair pathways may play a key role in oocyte aging (Titus et al., 2013). In addition, ATM-mediated DNA DSB repair function declines with age in human oocytes, resulting accumulation of DNA DSBs with age, rendering them more susceptible to genotoxic stress (Nicolai et al., 2015).

Although less obvious then in females, recent research indicate that men also show significant signs of reproductive aging. (Kong et al., 2012). Sperm counts show age-related decline and similar to women, chromosomal abnormalities and DNA fragmentation increase in sperm with age (Winkle et al., 2009). Sperm from older men, especially when female partner is also older than 40 years of age, contribute to infertility (Harris et al. 2011). Moreover, the incidence of schizophrenia, learning disorders and other conditions including autism, dwarfism, Apert Syndrome, (caused by FGFR2 mutations), achondroplasia, and thanatophoric dysplasia (FGFR3), and Costello syndrome (HRAS), collectively term “paternal age effect” (PAE) disorders are increased among children born from older men as men passes on more mutations to his offspring (Goriely et al., 2012). With a significant trend in delaying childbearing both in men and women (Chandra et al., 2013), male reproductive aging could adversely affect an increasing proportion of children being born. Despite its prominent public health impact, little is known on the mechanism of male reproductive aging and methods to slow down this process.

In the studies supporting the embodiments of this patent application, the declining DNA DSB repair has a significant role in sperm aging, similar to its role in oocytes. Furthermore we showed that a ceramide-induced death inhibitor Sphingosine-1-phosphate (S1P) reversed sperm aging process and improved its quality. S1P has been shown to reduce chemotherapy and radiotherapy-induced oocyte death but the mechanism of its action is not completely understood. We previously showed a common mechanism between chemotherapy- and age-induced oocyte death via the induction of DNA DSBs, the examples presented in this patent application show that S1P reduces gamete aging by enhancing DNA DSB repair.

In certain embodiments, S1P, a pharmaceutically acceptable salt thereof, or an analog thereof disclosed herein is used to enhance DNA repair and treat conditions that are associated with DNA repair deficiency and prevent such conditions. In certain embodiments, the conditions includes, but are not limited to, cancer, premature aging, organ failure, infertility, neurovascular degeneration, Alzheimer's, cardiac or pulmonary insufficiency and other systemic failures and diseases. In certain embodiments, such conditions or diseases include presence of BRCA1 and BRCA2 mutations, Fanconi Anemia or presence of Fanconi mutations, P53 mutations, Bloom Syndrome, Ataxia Telengiectiasia-Mutated Disorder, Cockayne's syndrome, Progeria (Hutchinson-Gilford Progeria syndrome), Rothmund-Thompson syndrome, Trichothiodystrophy, Werner Syndrome, Xeroderma Pigmentosum, UV-sensitive syndrome (The responsible genes are CSA, CSB or UVSSA). Because one of the major consequences of defective DNA repair and aging is increased cancer risk, in one embodiment, S1P, a pharmaceutically acceptable salt thereof, or an analog thereof is used in a method of preventing cancer. The table below shows examples of DNA repair gene defects which result in increased risk in numerous cancers.

TABLE 1
DNA Repair Gene Mutations and Associated Increase in Cancer Risk

| DNA Repair Gene | Proteins Affected | Mechanism Affected | Cancer with Increased Risk |
|---|---|---|---|
| RECQL4 | RECQ4 | Helicase likely active | basal cell carcinoma, |
| | | in HRR³² | squamous cell carcinoma, |
| | | | intraepidermal |
| | | | carcinoma¹ |
| Nijmegen breakage | NBS (NBN) | NHEJ¹⁰ | lymphoid cancers¹⁰ |
| syndrome | | | |
| mutY homolog | MUTYH | BER of A paired with | Colon²³ |
| (E. coli) | | 8-oxo-dG²³ | |
| mutS (E. coli) | MSH2 MSH6 ML | MMR²⁴ | colorectal, endometrial²⁴ |
| homolog 2, mutS | H1PMS2 | | |
| (E. coli) homolog 6, | | | |
| mutL (E. coli) | | | |
| homolog 1, postmeiotic | | | |
| segregation increased 2 | | | |
| (S. cerevisiae) | | | |
| MRE11A | MRE11 | HRR and NHEJ³⁰ | Breast⁴ |
| Fanconi anemia genes | FANCA etc. | HRR and TLS³³ | leukemia, liver tumors, |
| FANCA, B, C, D1, D2, | | | solid tumors many |
| E, F, G, I, J, L, M, N | | | areas² |
| breast cancer 1 & 2 | BRCA1 BRCA2 | HRR of double strand breaks | breast, ovarian²¹ |
| | | and daughter strand gap²⁶ | |
| Bloom syndrome | BLM (helicase) | HRR²⁸ | leukemia, lymphoma, |
| | | | colon, breast, skin, lung, |
| | | | auditory canal, tongue, |
| | | | esophagus, stomach, |
| | | | tonsil, larynx, uterus¹² |
| ataxia | ATM | Different mutations | leukemia, lymphoma, |
| telangiectasia | | in ATM reduce HRR, | breast^{18,34} |
| mutated | | SSA or NHEJ¹⁸ | |

In certain embodiments, S1P, a pharmaceutically acceptable salt thereof, or an analog thereof with DNA repair enhancing abilities is administered on a continuous, semi-continuous or intermittent basis via various routes. In certain embodiments, the routes include oral, intravenous, transdermal, intramuscular, intranasal, rectal, intraurethral, direct injection to any organ, vaginally, intraperitoneally or via any means rendering systemic absorption depending on the underlying DNA repair deficiency condition, risk of cancer and other individual factors. In certain embodiments, the duration of treatment is 1-5 days, 5-10 days, 10-20 days, 20-30 days, 1-2 months, 2-4 months, 4-6 months, 6-12 months, 1-3 years, 3-6 years, 6-10 years, 10-20 years, 20-30 years, 30-40 years or 40-60 years. The dose may be lower for life time administration and may be modified depending on the administration route or the potency of the salt or analog. In one embodiment, suitable local concentration of S1P, a pharmaceutical acceptable salt thereof, or an analog is 1-25, 25-50, 50-100, 100-150, 150-200, 200-250, 250-300, 300-350, 350-400, 400-450 or 450-500 μM.

In certain embodiments, suitable dosage ranges for oral administration are generally about 0.001 milligram to 200 milligrams of S1P, a pharmaceutical acceptable salt thereof, or an analog thereof per kilogram body weight. In specific preferred embodiments, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. Oral compositions preferably contain 10% to 95% active ingredient by weight.

In certain embodiments, suitable dosage ranges for intravenous administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams per kilogram body weight and comprise active ingredient in the range of 0.5% to 10% by weight. Recommended dosages for transdermal, intramuscular, intraperitoneal, subcutaneous, intravaginal administration or administration by inhalation are in the range of 0.001 milligram to 200 milligrams per kilogram of body weight. Effective doses may be extrapolated from dose-response curves derived from in vitro or animal model test systems. Such animal models and systems are well known in the art.

The acid sphigomyelinase (ASMase) antagonizers such as S1P or S1P analogs and derivatives, may include any compound, that suppresses or inhibits activity and/or expression of one or more acid sphingomylinase gene products and/or enhance DNA repair in vitro, ex vivo, or in vivo. The agent comprises, for example, any lipid, lysophospholipid, sphingolipid, protein, peptide, polypeptide, nucleic acid molecule, including DNA, RNA, DNA/RNA hybrids or an antisense molecule, small molecules, antibiotics, and the like. The terms protein, peptide, and polypeptide are used interchangeably herein.

A preferred agent according to the invention is a small molecule. In a more preferred embodiment of the invention, the agent comprises lysophospholipids, and most preferably, the agent is sphingosine-1-phosphate (S1P), a pharmaceutically acceptable salt thereof, or an analog thereof. Examples of analogs of sphingosine-1-phosphate, include but are not limited to, N,N-dimethylsphingosine-1-phosphate; N,N,N-trimethylsphingosine-1-phosphate; N-acetyl sphingosine-1-phosphate; N-acylsphingosine-1-phosphate; sphingosine-1,3-diphosphate; sphingosine-3-phosphate; sphingosine-1-thiophosphate; N,N-dimethylsphingosine-1-thiophosphate; N,N,N-trimethylsphingosine-1-thiophosphate; or pharmaceutically acceptable salts thereof as well as any or all of S1P receptor analogs.

Sphingosine-1-phosphate is shown to be safe and without side effects on the testes and sperm. It is noted that, in vivo continuous or intermittent administration of the agent to mice resulted in no detectable side effects (Soleimani et al., 2011; Li et al, 2014). In addition, an S1P analog, FTY720 has been approved by the FDA for the treatment of multiple sclerosis and showed acceptable safety profile in clinical use. It is marketed under the pharmacological name of Fingolimod and trade name of Gilenya, and its patent is held by Novartis (http://www.businesstoday.in/sectors/pharma/torrent-wins-patent-battle-against-novartis-usd-2.5-billion-drug-gilenya-in-us/story/224308.html). Fingolimod is used in the treatment of the relapsing form of multiple sclerosis. Its effect in those with primary progressive MS is not clear. It is also used in chronic inflammatory demyelinating polyneuropathy (Ayzenberg et al., 2016). It was originally proposed as an antirejection medication indicated after transplantation but it failed to show any significant benefit in post-transplantation clinical trials.

### ABBREVIATIONS

ASMase: acid sphingomyelinase (ASMase) antagonist;

AO staining: Acridine orange staining;

ATM: ataxia-telangiectasia mutated; and ATM inhibitors ATM and ATR are members of the phosphatidylinositol 3-kinase-related kinase (PIKK) family of serine/threonine protein kinases, which also comprises DNA-dependent protein kinase catalytic subunit (DNA-PKcs/PRKDC), mammalian target of rapamycin (MTOR/FRAP) and suppressor of morphogenesis in genitalia (SMG1). The cellular functions of these protein kinases range from regulation of the DNA damage response (DDR) to cell survival, proliferation, metabolism, differentiation, motility and nonsense-mediated mRNA decay (Lempiäinen & Halazonetis, Emerging common themes in regulation of PIKKs and PI3Ks EMBO J, 28 (2009), pp. 3067-3073).

DSB: double strand break;

FLICA: fluorescein-labelled inhibitors of caspases;

5FU: 5-fluorouracil;

GIFT: gamete intrafallopian transfer;

ICSI: intracytoplasmic sperm injection;

ZIFT: zygote intrafallopian transfer;

S1P: sphingosine 1 phosphate.

TESE: testicular sperm extraction;

TESA: testicular sperm aspiration;

microTESE: microscopic testicular sperm extraction.

In accordance with the present disclosure, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual, 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken. N.J.; and Enna et al. eds. (2005) Current Protocols in Pharmacology, John Wiley and Sons, Inc.: Hoboken, N.J.

In accordance with the present invention there may be employed conventional molecular biology, microbiology, protein expression and purification, antibody, and recombinant DNA techniques within the skill of the art. Such techniques are explained fully in the literature. See, e.g., Sambrook, Fritsch and Maniatis. Molecular Cloning: A Laboratory Manual, Second Edition (Cold Spring Harbor Laboratory Press, New York: 1989); DNA Cloning: A Practical Approach, Volumes I and II (Glover ed.: 1985); Oligonucleotide Synthesis (Gait ed.: 1984); Nucleic Acid Hybridization (Hames & Higgins eds.: 1985); Transcription And Translation (Hames & Higgins, eds.: 1984); Animal Cell Culture (Freshney, ed.: 1986); Immobilized Cells And Enzymes (IRL Press: 1986); Perbal, A Practical Guide To Molecular Cloning (1984); Ausubel et al., eds. Current Protocols in Molecular Biology, (John Wiley & Sons, Inc.: 1994); and Harlow and Lane. Antibodies: A Laboratory Manual (Cold Spring Harbor Laboratory Press: 1988).

By “nucleic acid” or “nucleic acid molecule” is meant to include a DNA, RNA, mRNA, cDNA, or recombinant DNA or RNA.

By “animal” is meant any member of the animal kingdom including vertebrates (e.g., frogs, salamanders, chickens, or horses) and invertebrates (e.g., worms, etc.). Preferred animals are mammals. Preferred mammalian animals include livestock animals (e.g., ungulates, such as bovines, buffalo, equines, ovines, porcines and caprines), as well as rodents (e.g., mice, hamsters, rats and guinea pigs), canines, felines and primates. By “non-human” is meant to include all animals, especially mammals and including primates other than human primates.

By “female surrogate” is meant a female animal into which an embryo of the invention is inserted for gestation. Typically, the female animal is of the same animal species as the embryo, but the female surrogate may also be of a different animal species. The embryo, as used herein, can include a complex of two or more cells.

By “cytoplast” is meant the fragment of the cell remaining once the nucleus is removed.

By “parthenogenetic activation” is meant development of an ovum or oocyte without fusion of its nucleus with a male nucleus or male cell to form a zygote. By “oocyte” is meant an animal egg, nucleated or enucleated which has not undergone a Ca²⁺ oscillations. By “activated oocyte” is meant an oocyte which acts as though it has been parthenogenically activated or as though it has been fertilized. By “enucleated oocyte” is meant an animal egg which has had its endogenous nucleus removed or inactivated.

By “sperm,” “semen,” “sperm sample,” and “semen sample” are meant the ejaculate from a male animal which contains spermatozoa. A mature sperm cell is a “spermatozoon,” whereas the precursor is a “spermatid.” Spermatids are the haploid products of the second meiotic division in spermatogenesis, which differentiate into spermatozoa.

By testicles (or testes) are meant oval organs about the size of large olives that lie in the scrotum, secured at either end by a structure called the spermatic cord. Most men have two testes. The testes are responsible for making testosterone, the primary male sex hormone, and for generating sperm. Within the testes are coiled masses of tubes called seminiferous tubules. These tubes are responsible for producing sperm cells.

By “sperm fertility” is meant the ability of a sperm to fertilize an egg and create an embryo. By “sperm [Ca²⁺]ᵢ-releasing activity” is meant the ability of a sperm to activate an oocyte (of any animal), which can be measured by induction of Ca²⁺ oscillations in the oocyte.

By “sperm cytoplasmic fraction” is meant the portion of the cell which lacks the nucleus and most of the genetic material. Preferably, the cytoplasm fraction comprises the substances contained within the plasma membrane but excluding the nucleus and its genetic material.

By “inducing”, “increasing,” “enhancing” or “up-regulating” is meant the ability to raise the level of a desired activity. By “enhancing activation” is meant a method or agent which increases a desired activation.

By “modulating” or “regulating” is meant the ability of an agent to alter. (e.g., up-regulate or down-regulate) from the wild type level observed in the individual organism the activity of a specified nucleic acid, protein, or other activity. Such activity can be at the level of transcription, translation, nucleic acid or protein stability or protein activity.

By “intracytoplasmic sperm injection” or “ICSI” is meant injection of a sperm or at least the genetic contents of a sperm into an oocyte.

The terms “nuclear transfer” or “nuclear transplantation” refer to a method of cloning, wherein the donor cell nucleus is transplanted into a cell before or after removal of its endogenous nucleus. The cytoplast could be from an enucleated oocyte, an enucleated ES cell, an enucleated EG cell, an enucleated embryonic cell or an enucleated somatic cell. Nuclear transfer techniques or nuclear transplantation techniques are known in the literature (Campbell et al., Theriogenology 43: 181 (1995); Collas et al., Mol. Reprod. Dev. 38: 264-267 (1994); Keefer et al., Biol. Reprod. 50: 935-939 (1994); Sims et al., Proc. Natl. Acad. Sci. USA 90: 6143-6147 (1993): Evans et al., WO 90/03432; Smith et al., WO 94/24274; and Wheeler et al., WO 94/26884. Also U.S. Pat. Nos. 4,994,384 and 5,057,420 describe procedures for bovine nuclear transplantation. In the subject application, “nuclear transfer” or “nuclear transplantation” or “NT” are used interchangeably.

The terms “nuclear transfer unit” and “NT unit” refer to the product of fusion between or injection of a somatic cell or cell nucleus and an enucleated cytoplast (e.g., an enucleated oocyte), which is some-times referred to herein as a fused NT unit.

By “somatic cell” is meant any cell of a multicellular organism, preferably an animal, that does not become a gamete.

By “differentiate” or “differentiation” is meant to refer to the process in development of an organism by which cells become specialized for particular functions. Differentiation requires that there is selective expression of portions of the genome.

By “inner cell mass” or “ICM” is meant a group of cells found in the mammalian blastocyst that give rise to the embryo and are potentially capable of forming all tissues, embryonic and extra-embryonic, except the trophoblast.

By “feeder layer” is meant a layer of cells to condition the medium in order to culture other cells, particularly to culture those cells at low or clonal density.

By “medium” or “media” is meant the nutrient solution in which cells and tissues are grown.

The term “pharmaceutically acceptable carrier”, as used herein means a pharmaceutically-acceptable material, composition or vehicle, such as a liquid or solid filler, diluent, excipient, solvent or encapsulating material, involved in carrying or transporting a chemical agent. The diluent or carrier ingredients should not be such as to diminish the therapeutic effects of the active compound(s).

The term “composition” as used herein means a product which results from the mixing or combining of more than one element or ingredient.

“Treating” or “treatment” of a state, disorder or condition includes:

(1) preventing or delaying the appearance of clinical symptoms of the state, disorder, or condition developing in a person who may be afflicted with or predisposed to the state, disorder or condition but does not yet experience or display clinical symptoms of the state, disorder or condition; or

(2) inhibiting the state, disorder or condition. i.e., arresting, reducing or delaying the development of the disease or a relapse thereof (in case of maintenance treatment) or at least one clinical symptom, sign, or test, thereof; or

(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the animal to be treated.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit, 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

“Patient” or “subject” refers to mammals and includes human and veterinary subjects. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

The dosage of the therapeutic S1P or stabilized sperm formulations will vary widely, depending upon the nature of the patient, the potential disease, insult, or disorder, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park. Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, and BCG (bacille Calmette-Guerin). Preferably, the adjuvant is pharmaceutically acceptable.

In the case of the present invention, a number of alternative routes of administration of therapeutic or stabilized sperm formulations are also possible. Such routes include, vaginal, intraurethral, or intratesticular routes.

### Kits

In another aspect, the present invention provides any of the compositions, therapeutic or stabilized sperm formulations, described herein as a kit, optionally including instructions for use of the compositions (e.g., for preserving sperm and/or other cells). That is, the kit can include a description of use of a composition in any method described herein. A “kit.” as used herein, typically defines a package, assembly, or container (such as an insulated container) including one or more of the components of the invention, and/or other components associated with the invention, for example, as previously described. Each of the components of the kit may be provided in liquid form (e.g., in solution), or in solid form (e.g., a dried powder, frozen, etc.).

In some cases, the kit includes one or more components, which may be within the same or in two or more receptacles, and/or in any combination thereof. The receptacle is able to contain a liquid, and non-limiting examples include bottles, vials, jars, tubes, flasks, beakers, or the like. In some cases, the receptacle is spill-proof (when closed, liquid cannot exit the receptacle, regardless of orientation of the receptacle).

In some embodiments, a kit may comprise two or more of: SP1, a membrane protectant, a free radical scavenger, and a cryoprotective agent, which may be in the same receptacle, or divided among two or more receptacles. As a specific, non-limiting example, a first receptacle may contain at least two of: the membrane protectant, the free radical scavenger, and the cryoprotective agent, while a second receptacle may contain a component that is not present in the first vessel. In some cases, the components of the kit may be contained within a suitable container, such as a cardboard box, a Styrofoam box, etc. The kit may be shipped at room temperature (about 25° C.), chilled (e.g., at about 4° C.), and/or any one or more of the components may be shipped frozen (e.g., between −20° C. and −80° C., at about −150° C., etc.) or in liquid nitrogen (about −196° C.). In some cases, one or more of the components are frozen and/or shipped on dry ice (about −80° C.). Options for cryopreserving sperm are described in U.S. Pat. Nos. 8,623,658; 8,435,729; and 8,685,637.

With regard to frozen solutions, if more than one component is present (e.g., as described above), the components may be frozen together in one common liquid (e.g., within one common receptacle), or as two or more separate liquids (e.g., within separate receptacles).

In certain cases, some of the components may be processable (e.g., to an active form), for example, by the addition of a suitable solvent or other species, which may or may not be provided with the kit. For example, the component may be heated or a liquid may be added to the component (e.g., if the component is frozen, lyophilized, shipped in a concentrated form, etc.).

In some cases, the kit will include a vessel suitable for containing materials at vitrification temperatures, for example, liquid nitrogen. Those of ordinary skill in the art will be aware of suitable cryogenic vessels, for example, a Dewar flask (e.g., formed from stainless steel and/or aluminum, etc.), a vapor shipper, a stainless steel container, a Styrofoam container, or the like. Typically, cryogenic temperatures include temperatures below about −150° C. below about −170° C., or below about −190° C. For instance, liquid nitrogen has a boiling point of about −196° C.

The kit may also contain a receptacle for holding sperm and/or other cells. In some cases, this receptacle is able to contain the liquid containing the sperm, and/or a frozen solution or liquid containing the sperm. For example, the receptacle may be constructed so that it can withstand cryogenic temperatures without rupture or fracture. In some embodiments, the receptacle can be placed within a cryogenic vessel, as described above, (e.g., using a float (for example, that can float on liquid nitrogen or other cryogenic liquid within the cryogenic vessel)). Non-limiting examples of receptacles for sperm and/or other suitable cells include cell straws, glass ampoules, cryotubes, cryovials, etc. The receptacle may be pre-labeled in certain instances.

Examples of other compositions or components associated with the invention include, but are not limited to, diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

A kit of the invention generally will include instructions or instructions to a website or other source in any form that are provided for using the kit in connection with the components and/or methods of the invention. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping at room temperature, sub-zero temperatures, cryogenic temperatures, etc. The instructions may be provided in any form that is useful to the user of the kit, such as written or oral (e.g., telephonic), digital, optical, visual (e.g., videotape, DVD, etc.) and/or electronic communications (including Internet or web-based communications), provided in any manner.

As used herein, instructions can include protocols, directions, guides, warnings, labels, notes, and/or “frequently asked questions” (FAQs), and typically involve written instructions on or associated with the invention and/or with the packaging of the invention. Instructions can also include instructional communications in any form (e.g., oral, electronic, digital, optical, visual, etc.), provided in any manner (e.g., within or separate from a kit) such that a user will clearly recognize that the instructions are to be used with the kit.

As an example, a kit as discussed herein may be shipped to a user, typically with instructions for use. For instance, the instructions may instruct the user to add sperm to the membrane protectant and the free radical scavenger, and store the resulting combination and/or return the kit and the sperm to the sender. As another example, the instructions may instruct the user to combine of sperm, membrane protectant, free radical scavenger, and cryoprotective agent, and cryopreserve the resulting combination (e.g., as described above). The combination could then be stored, returned to the shipper for storage and later recovery, or the like. In one embodiment, the kit may include the partner's processed sperm which would be mixed with S1P and a radical scavenger, prior to using a vaginal injector for self-intravaginal insemination.

### Examples

The following examples are included to demonstrate certain embodiments of the invention. These specific examples are described solely for purposes of illustration, and are not intended to limit the scope of this disclosure. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. Although specific targets, terms, and values have been employed herein, such targets, terms, and values will likewise be understood as exemplary and non-limiting to the scope of this invention.

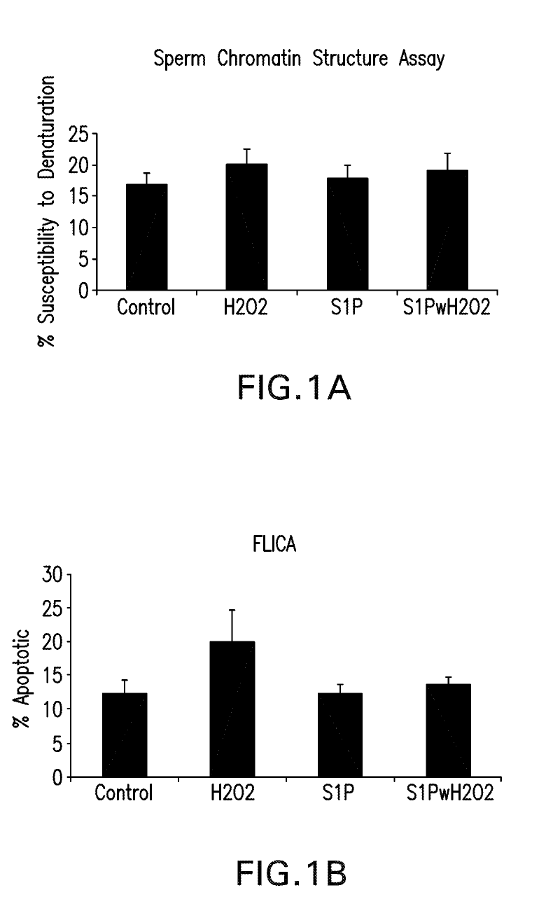

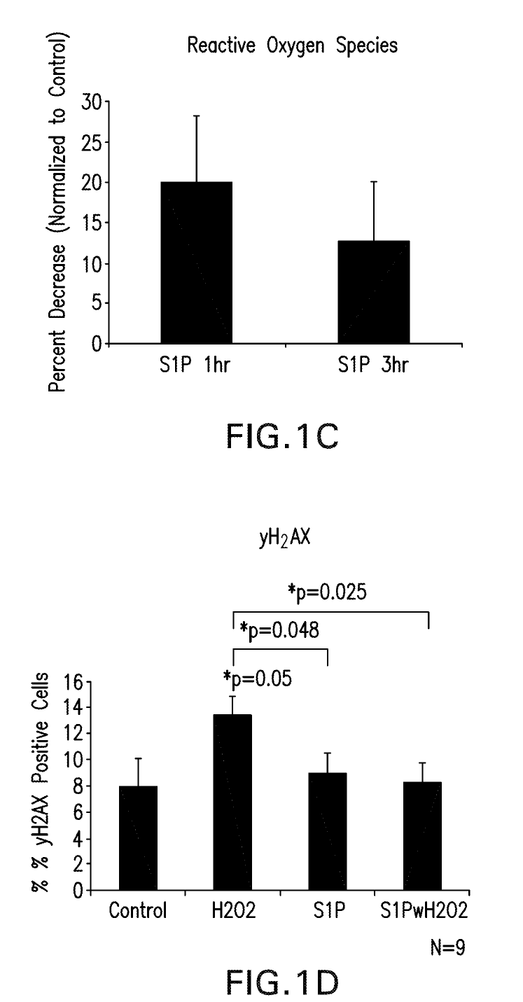

To demonstrate that S1P enhances DNA DSB repair and reduces sperm aging, S1P-treated mouse sperm were subjected to genotoxic stress with and without S1P pre-treatment in vitro and DNA damage, apoptosis, chromatin integrity, and reactive oxygen species were determined. FIGS. 1A-D illustrate a significant decrease in DNA DSBs as reported by γH2AX expression when S1P was administered in the presence of genotoxic stress (8.29±1.45 vs. 13.42±1.51, n=9; p=0.025) and S1P alone (8.94±1.48 vs. 13.42±1.51, n=9; p=0.048), as compared to the genotoxic stress alone. As expected, genotoxic stress alone caused an increase in DNA damage as compared to the control (13.42±1.51 vs. 7.93±2.2, n=9; p=0.05). There was no significant difference in the percentage of apoptosis by FLICA, as well as susceptibility of DNA to denaturation by AO staining, and reactive oxygen species as a result of S1P treatment (FIGS. 1A-D). *P<0.01, Student's t test. All results are mean±SEM.

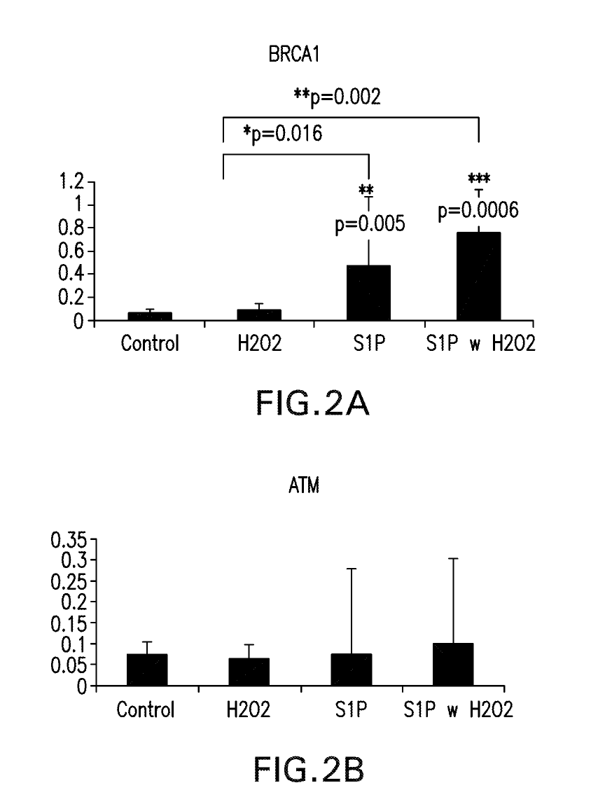

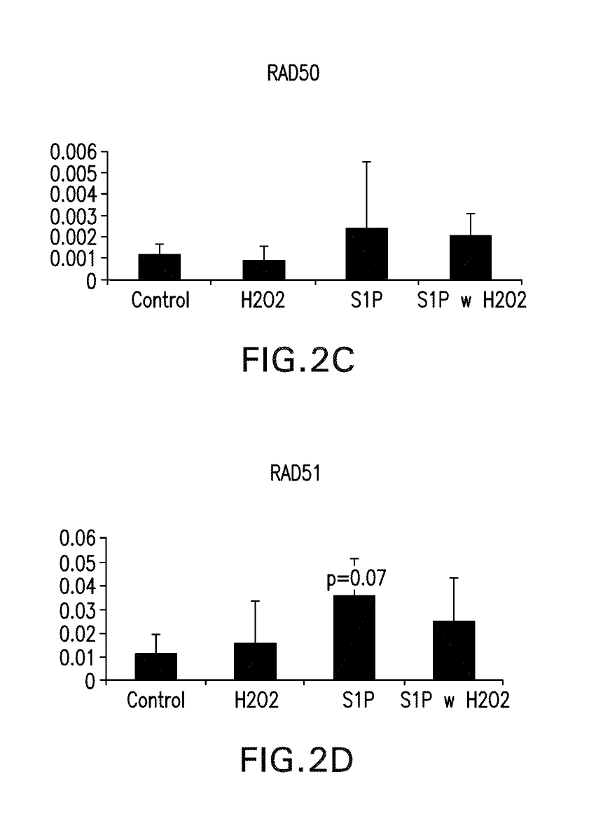

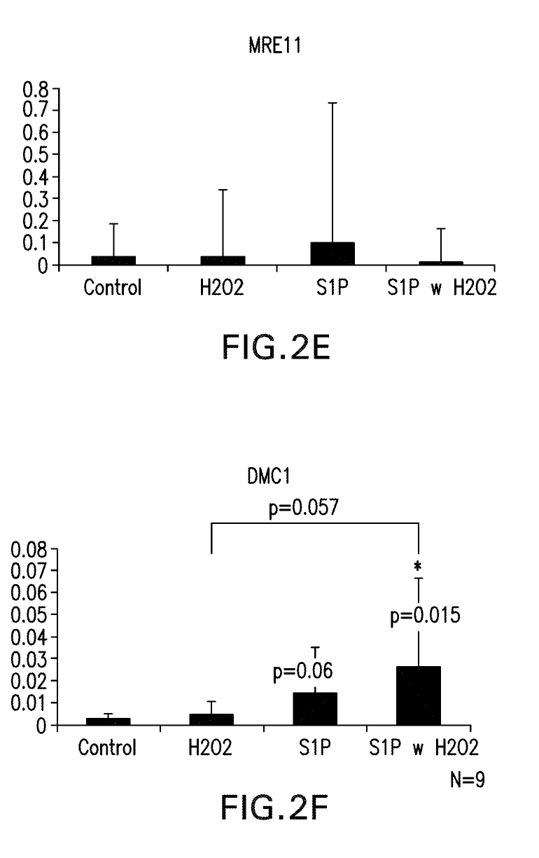

The DNA DSB repair gene expression was analyzed by qRT-PCR in sperm lysates to determine whether S1P protects gametes by enhancing DNA repair. The results shown in FIGS. 2A-F demonstrate an increase in the expression of key DNA repair genes was observed, including BRCA1 when S1P was administered in the presence of genotoxic stress (0.755±0.378 vs. 0.068±0.031, n=9; p=0.0006) and S1P alone (0.471±0.602 vs. 0.068±0.031, n=9; p=0.005), as compared to control. In addition, there was an increase in BRCA1 after S1P treatment with genotoxic stress (0.755±0.378 vs. 0.090±0.053, n=9; p=0.002) and S1P alone (0.471±0.602 vs. 0.090±0.053, n=9; p=0.016), as compared to genotoxic stress alone. DMC1, another ATM-mediated DNA double strand break repair gene, also showed increased expression in sperm after treatment with S1P in the presence of genotoxic stress (0.029±0.04 vs. 0.004±0.002, n=9; p=0.015) as compared to control (FIGS. 2A-F). Other DNA repair genes including RAD50, RAD51, ATM, and MRE11 were tested and showed no significant difference. *P<0.01, Student's t test. All results are mean±SEM.

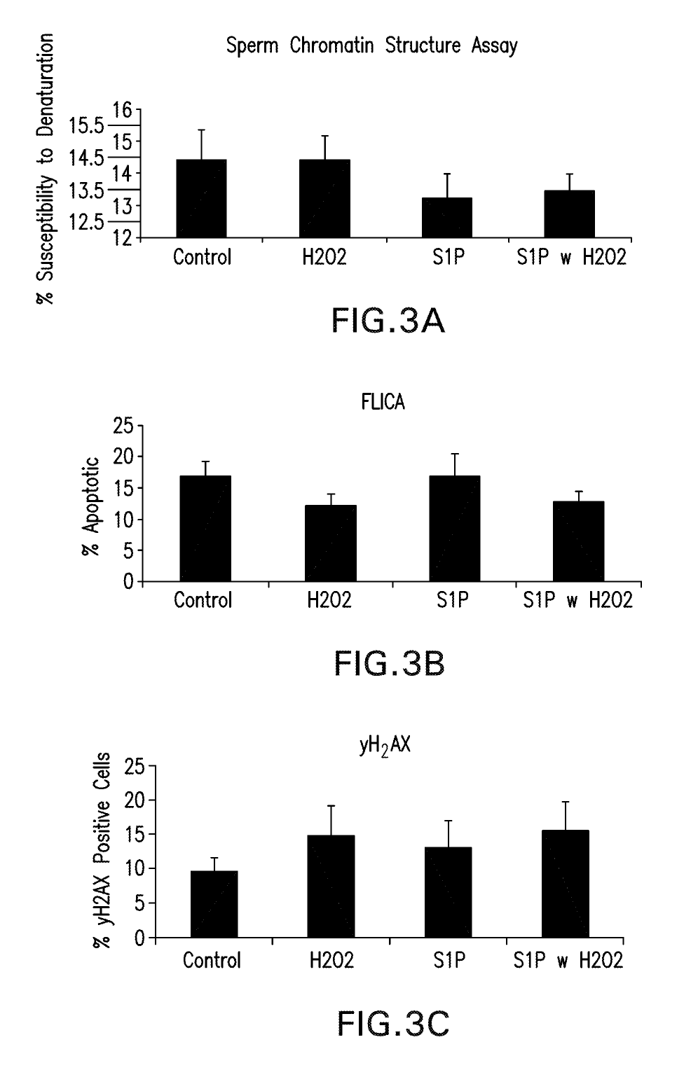

In addition, to determine whether S1P reduced DNA damage by directly acting on the ATM-mediated DNA DSB repair pathway, previous experiments were also repeated in the presence or absence of an ATM inhibitor. As shown in FIGS. 3A-C ATM inhibition reversed the protective effects of S1P on DNA integrity (n=9), indicating that S1P reduces DNA damage by its direct enhancive actions on the ATM-mediated double strand DNA break (DSB) repair pathway. This discovery represents a novel and paradigm changing breakthrough as this is the first ever treatment found to enhance DNA repair.

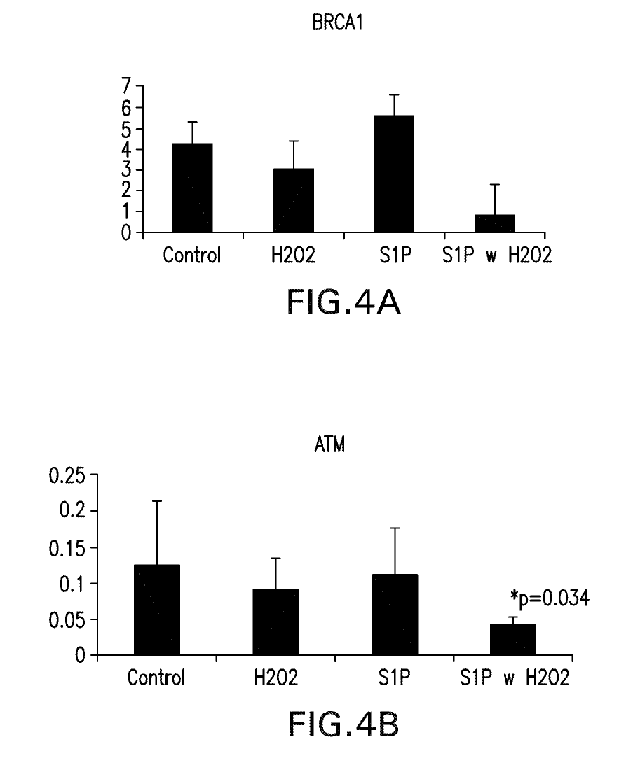

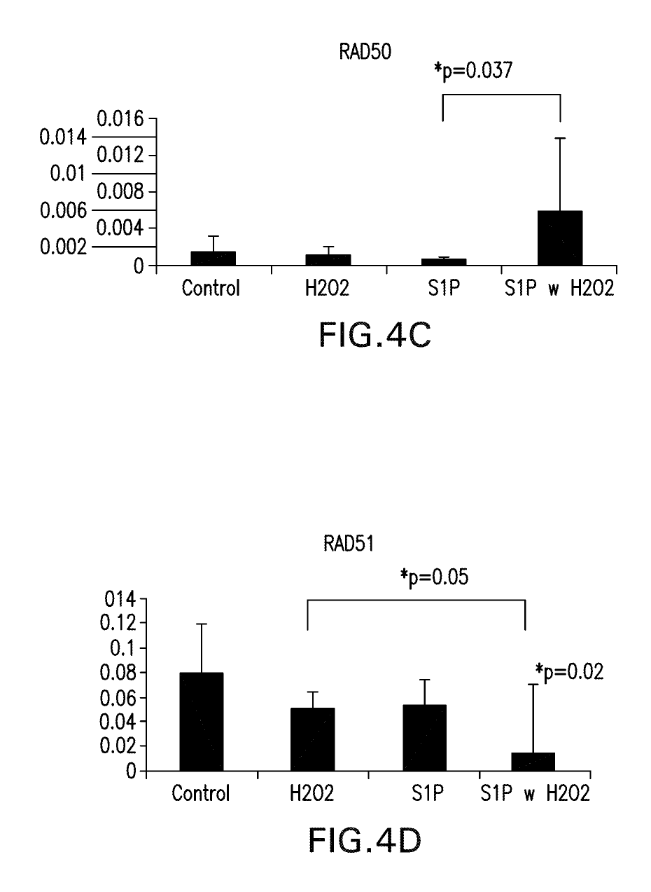

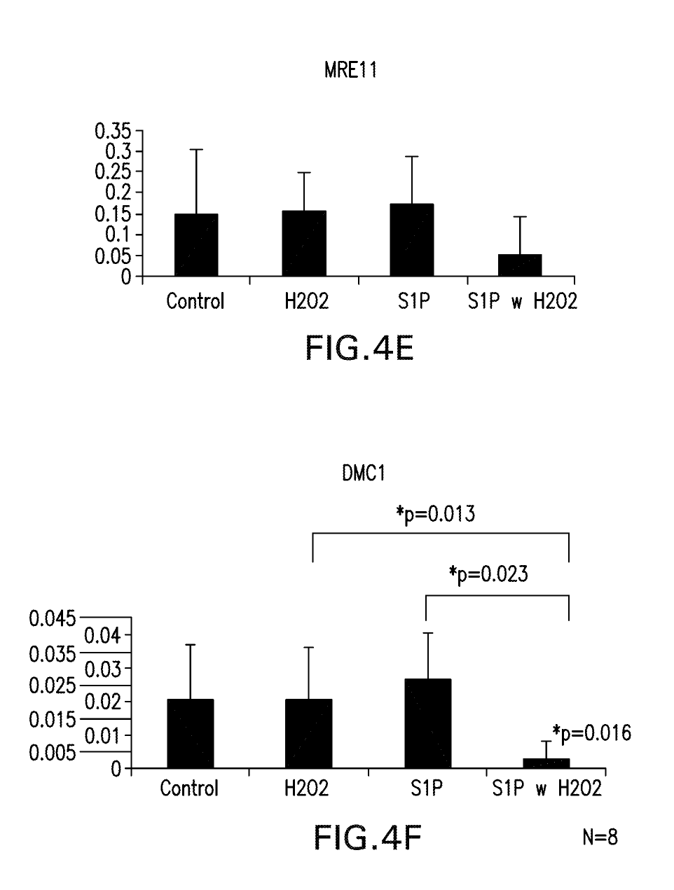

To further prove that S1P enhances DNA repair via its direct effects of the function of ATM-mediated DNA repair genes, we looked at the impact of S1P on the expression of the said genes with and without an ATM inhibitor. Strikingly and FIGS. 4A-F illustrate, ATM inhibition reversed the positive effects of S1P treatment on the expression of ATM-mediated DNA repair genes. These included reduction of the expression of the ATM gene after S1P treatment with genotoxic stress as compared to control (0.042±0.011 vs. 0.125±0.089, n=8; p=0.034), the expression of RAD51 after S1P treatment with genotoxic stress as compared to control (0.015±0.056 vs. 0.08±0.039, n=8; p=0.02) and as compared to genotoxic stress alone (0.015±0.056 vs. 0.052±0.012, n=8; p=0.05), and the expression of DMC1 after S1P treatment with genotoxic stress as compared to control (0.0035±0.005 vs. 0.021±0.016, n=8; p=0.016), S1P alone (0.0035±0.005 vs. 0.027±0.014, n=8; p=0.023) and genotoxic stress alone (0.0035±0.005 vs. 0.021±0.015, n=8; p=0.013). ATM inhibition also blocked the S1P induced increase in the expression of BRCA1 and MRE11 were tested and showed no significant difference. (In FIG. 4: *P<0.01, Student's t test. All results are mean±SEM)

To confirm these results with an independent method of DNA damage assessment, both alkaline and neutral comet assay were conducted on S1P-treated mouse sperm with and without an ATM inhibitor. In COMET assay, the percent tail DNA is determined as it corresponds to the amount of damage in the sample. Alkaline comet S1P-treated mouse sperm without ATM inhibitor showed a significant increase in DNA damage in H₂O₂ (27.3±0.90 vs. 24.7±0.76, n=9; p=0.045), and decrease in S1P alone (16.79±0.89 vs. 24.7±0.76, n=9; p=0.000093), and S1P with H₂O₂ group (16.8±0.87 vs. 24.7±0.76, n=9; p=0.00005), as compared to control. In addition, there was a significant decrease in S1P alone (16.79±0.89 vs. 27.3±0.90, n=9; p=0.00001), and S1P with H₂O₂ group (16.8±0.87 vs. 27.3±0.90, n=9; p=0.000094), as compared to H₂O₂ alone. By Neutral COMET, S1P-treated mouse sperm without ATM inhibitor showed a significant increase in DNA damage in H₂O₂ (29.6±0.64 vs. 27.8±0.52, n=9; p=0.028), and decrease in S1P alone (18.5±0.59 vs. 27.8±0.52, n=9; p=0.000018), and S1P with H₂O₂ group (18.8±0.73 vs. 27.8±0.52, n=9; p=0.000024), as compared to control. In addition, there was a significant decrease in DNA damage when S1P was used alone (18.5±0.59 vs. 29.6±0.64, n=9; p=0.000025), and S1P with H₂O₂ group (18.8±0.73 vs. 29.6±0.64, n=9; p=0.00004), as compared to H₂O₂ alone.

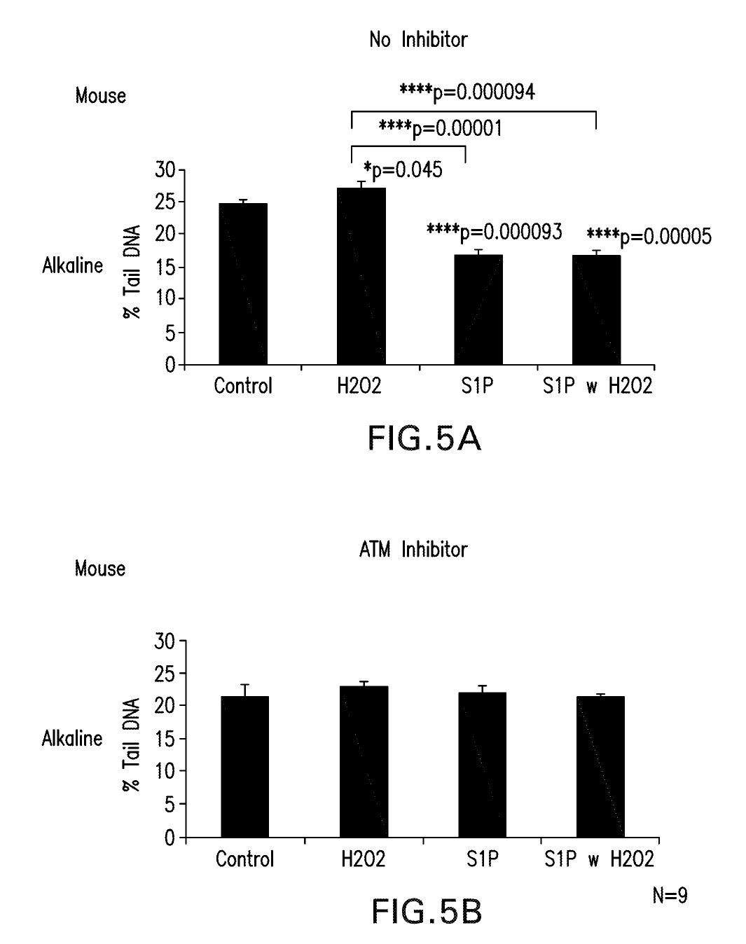

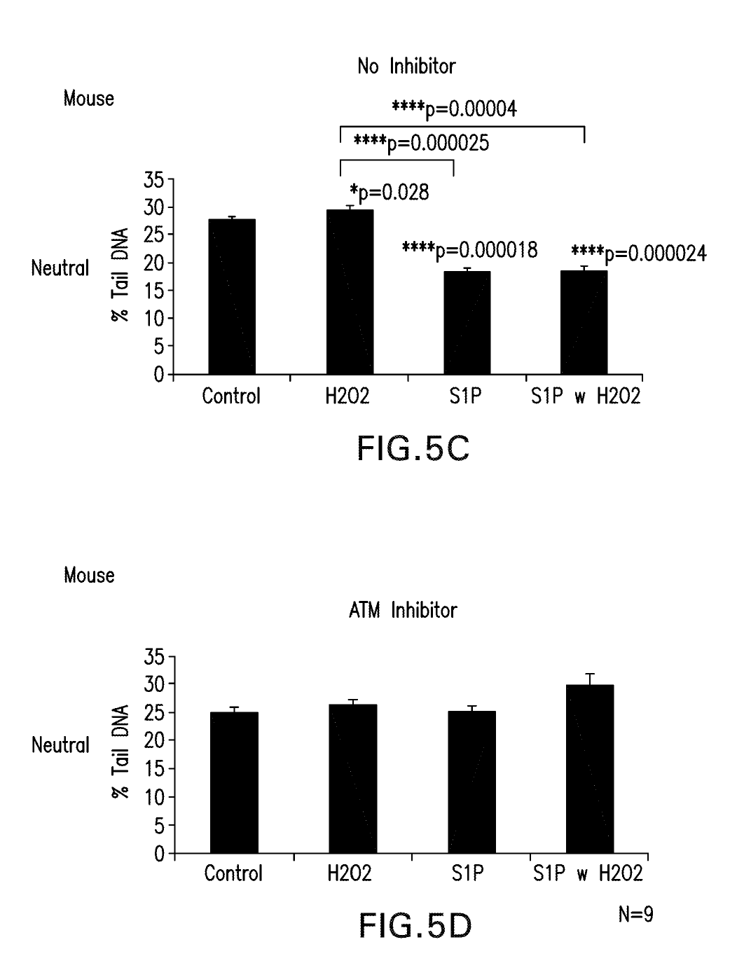

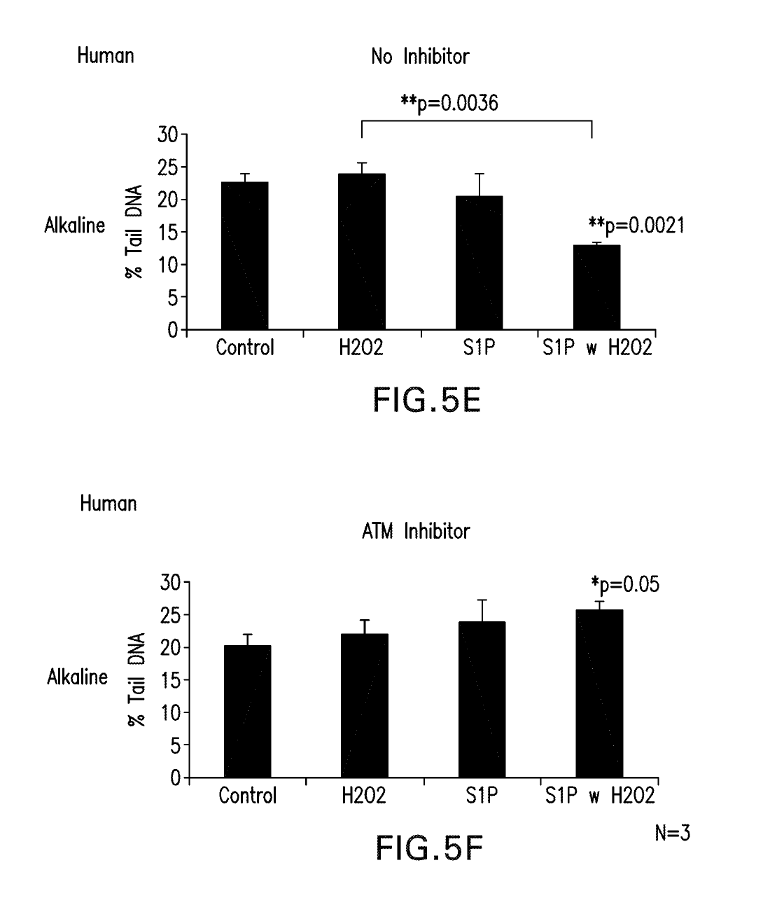

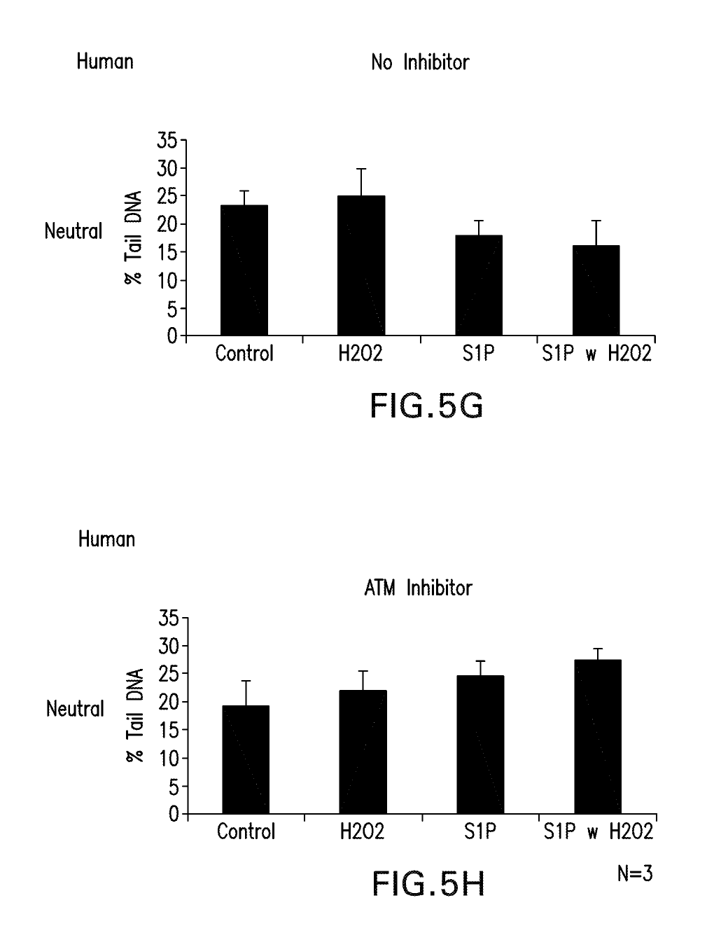

In order to translate our findings, both alkaline and neutral comet assay were performed on S1P-treated human sperm with and without an ATM inhibitor. The Bar graphs in FIGS. 5A-H show percent tail DNA for alkaline and neutral comet assay in S1P-treated mouse and human sperm with and without ATM inhibitor. Alkaline comet S1P-treated mouse sperm without ATM inhibitor showed a significant increase in DNA damage in H₂O₂ (27.3±0.90 vs. 24.7±0.76, n=9; p=0.045), and decrease in S1P alone (16.79±0.89 vs. 24.7±0.76, n=9; p=0.000093), and S1P with H₂O₂ group (16.8±0.87 vs. 24.7±0.76, n=9; p=0.00005), as compared to control. In addition, there was a significant decrease in S1P alone (16.79±0.89 vs. 27.3±0.90, n=9; p=0.00001), and S1P with H₂O₂ group (16.8±0.87 vs. 27.3±0.90, n=9; p=0.000094), as compared to H₂O₂ alone.

Neutral S1P-treated mouse sperm without ATM inhibitor showed a significant increase in DNA damage in H₂O₂ (29.6±0.64 vs. 27.8±0.52, n=9; p=0.028), and decrease in S1P alone (18.5±0.59 vs. 27.8±0.52, n=9; p=0.000018), and S1P with H₂O₂ group (18.8±0.73 vs. 27.8±0.52, n=9; p=0.000024), as compared to control. In addition, there was a significant decrease in S1P alone (18.5±0.59 vs. 29.6±0.64, n=9; p=0.000025), and S1P with H₂O₂ group (18.8±0.73 vs. 29.6±0.64, n=9; p=0.00004), as compared to H₂O₂ alone.

Results of alkaline and neutral comet assays on human sperm were also obtained. Evaluation of these images of alkaline and neutral comet assay in S1P-treated human sperm with and without ATM inhibitor showed that S1P treatment reduces DNA damage in human sperm, as well as in mouse sperm, as described above. These alkaline comet assays on S1P-treated human sperm without ATM inhibitor showed a significant decrease in DNA damage in the S1P with H₂O₂ group as compared to control (13.2±0.51 vs. 22.7±1.25, n=3; p=0.002) and H₂O₂ alone (13.2±1.25 vs. 24.0±1.70, n=3; p=0.003). Alkaline comet S1P-treated human sperm with ATM inhibitor showed significance increase in DNA damage in the S1P with H₂O₂ group as compared to control (26.1±1.19 vs. 20.4±1.76, n=3; p=0.05). There was no significant difference in alkaline and neutral comet for S1P-treated mouse sperm with ATM inhibitor and neutral comet for S1P-treated human sperm with and without ATM inhibitor. *P<0.01, Student's t test. All bar graphs show means±SEM.

These results show that there was a significant decrease in DNA DSB damage when sperm was treated with S1P under genotoxic stress. In contrast, based on the addition of an ATM inhibitor as shown herein, S1P likely exhibits its effect on DNA DSB repair through the ATM pathway, since as shown herein, the ATM inhibitor eliminated S1P's ability to prevent DNA DSB damage and increase gene expression of key DNA repair genes.

These data underscore the importance of DNA DSB repair in maintenance of gametes. In addition, the data suggests that S1P can improve DNA DSB repair in sperm which could potentially be used for delaying reproductive aging in males.

### Materials and Methods

The animal studies were carried out in strict accordance with the recommendations in the Guide for the Care and Use of Laboratory Animals of the U.S. National Institutes of Health (NIH) and the NYMC Institutional Animal Care and Use Committee at New York Medical College. All Friend's leukemia virus B (FVB) mice were purchased from Taconic. BRCA1 and mice were obtained from NCI Fredrick Mouse Repository. BRCA1 breeding colony was maintained and their offspring were genotyped.

### Mating & Genotyping

In the BRCA1 breeding colony, wild type female mice were co-housed with heterozygous male mice. The litter sizes produced by the mating setup were recorded. BRCA1 transgenic mice were selected on the basis of their sensitivity to genotoxic stress (Shen et al., 1998) and obtained from the NIH Fredrick Mouse Repository. The mice were kept in a temperature- and light-regulated room environment; 12-hour light/12-hour dark cycle and bred in-house. BRCA1-mutant mice carried a deletion of 330 base pairs (bp) in intron 10 plus 407 bp in exon 11 of the BRCA1 gene (BRCA1^{+/Δ11}). Owing to the deletion, the BRCA1^{Δ11} gene product is retained in the cytoplasm, which severely compromises its nuclear functions. BRCA1^{Δ11/Δ11} mice were not viable and died at embryonic days 7 to 8; BRCA1^{Δ11/Δ11} embryos exhibited both early post-implantation growth retardation and chromosomal abnormalities (Shen et al., 1998). γ-Irradiation-induced Rad51 focus formation is impaired in cells in which only BRCA1^{Δ11} was expressed (Huber et al., 2001).

After weaning, tail and ear biopsies were obtained and DNA was isolated for genotyping using the manufacturer's protocol. DNA was extracted by treating the biopsies with lysis buffer (Teknova) supplemented with proteinase K (Invitrogen) at 55° C. overnight. The following day, the DNA was precipitated with equal volumes of isopropanol. The pellet was washed in 70% ethanol and air-dried. After reconstitution with adequate amount of nuclease-free water, PCR was performed with gene-specific primers to identify the genotype (Table 1).

The primers used for identifying BRCA1 wild type were B004/B005, which amplifies a 450-bp fragment, and for BRCA heterozygous were B004/B007, which amplifies a 550-bp fragment (Shen et al., 1998). The PCR reaction mixture consisted of a 10 μl total volume of 10 μl, containing 5 μl ImmoMix (Bioline), 1 μl of primer (Integrated DNA Technologies), 3 μl of nuclease free water, and 1 μl of DNA. Cycling conditions were 94° C. for 5 minutes, 35 cycles of 94° C. for 0.5 minutes, 58° C. for 1 minute, and 72° C. for 45 seconds, followed by 72° C. for 10 minutes. Amplified products were run on 2% agarose gel electrophoresis, with ethidium bromide staining, and visualization with Alphalmager 2200 (Alpha Innotech) to determine the genotype.

### Collection and Processing of Mouse Spermatozoa and Testis

Young (2-3 month old) and reproductively senescing old (10-14 month) wild type (WT) male mice and young (2-3 month old) BRCA1-mutant mice were studied. Mice were euthanized via anesthesia and cervical dislocation. Testes were removed, fixed in 10% formaldehyde, embedded in paraffin, and serially sectioned for DNA damage assessment by immunohistochemistry analysis. The cauda epididymis and vas deferens were excised surgically and placed into a 35-mm dish (Becton Dickinson) with M199 media (Corning). Mature spermatozoa were collected by parallel action of applying gentle pressure with forceps at one end of vas deferens, and flushing with M199 media (Corning) with a 30-gauge needle. The needle was also used lengthwise along the epididymis to expel the sperm. Sperm were washed and manipulated using Multipurpose Handling Medium (Irvine Scientific). Sperm counts were calculated for each sample collected using a hemocytometer. Sperm were either studied live or fixed for immunocytochemistry and immunofluorescence studies.

### S1P Treatment

Treatment was performed with genotoxic stress (1 mM H₂O₂), S1P (200 μM), and S1P with genotoxic stress and control in culture for one hour. Also, the use of an ATM inhibitor was utilized (10 μM) (KU-55593, Abcam) (Li et al., 2014). We assessed DNA damage, apoptosis, and chromatin integrity. We also studied the DNA DSB repair gene expression by qRT-PCR in sperm lysates.

### Immunohistochemistry and Immunofluorescence

DNA damage assessment of testicular tissue from mice utilized a Ser139 phosphorylation-specific γH2AX antibody (IHC-00059; Bethyl Laboratories) performed with enzymatic diaminobenzidine staining (Vector). Histone H2AX, one of the several variants of the nucleosome core histone H2A, becomes phosphorylated on Ser139 in response to DSBs (γH2AX). Within seconds of DSB formation, γH2AX foci are formed at the site of DNA damage, which can be detected by confocal microscopy or immunohistochemistry to quantify DNA damage. Foci of γH2AX represent DDR in a 1:1 manner, enabling sensitive quantification of potential DSBs (Katsube et al., 2014). Primordial follicles containing a γH2AX-positive oocyte were considered to be DNA-damaged (Rogakou et al., 1998). Sperm were assessed for DNA damage with antibody to γH2AX (613402; BioLegend) and secondary antibody Alexa Fluor 488 (A-11029; Invitrogen), followed by nuclear counterstaining with diamidino-2-phenylindole (DAPI; Fisher Scientific). Immunofluorescence for SD sperm samples was measured by FACScan flow cytometer (Becton-Dickinson) or laser scanning cytometry (Halicka et al., 2008).

DNA integrity of mouse sperm were assessed via flow cytometry using acridine orange (AO) staining (Hawley and Hawley, 2004). AO fluoresces green when attached to native DNA, and fluoresces red when attached to fragmented DNA. Basically, after induction of denaturation by heat or acid the denatured DNA (ssDNA) stains metachromatically red with AO while dsDNA stains green. When the sample has ≥50% green fluorescence, the sample is of good quality. Similarly, AO was used to assess sperm chromatin structure by measuring its intensity of fluorescence, which is the ratio of red/(red+green) yields the percentage of DNA fragmentation (also known as DNA fragmentation index, or DFI %). Semen samples with sperm chromatin structure assay (SCSA) value of less than or equal to 15% DFI were regarded to have low level DNA fragmentation, samples having between 15% and less than or equal to 30% DFI value were regarded to have moderate DNA fragmentation, and samples having above 30% DFI were regarded as exhibiting high levels of DNA fragmentation (Evenson et al., 1980).

Levels of activated caspases were detected in mouse sperm samples using fluorescein-labelled inhibitors of caspases (FLICA). This cell-permeable and non-cytotoxic caspase inhibitor binds covalently to active caspases. The fluorogenic substrate becomes fluorescent on cleavage by the caspases. The inhibitors were used with the appropriate controls according to the kit instructions provided by the manufacturer (ImmunoChemistry Technologies). A 150-fold stock solution of the inhibitor was prepared in dimethylsulphoxide (DMSO). It was further diluted in phosphate-buffered saline (PBS) to yield a 30-fold working solution. Sperm samples (300 μl) were incubated at 37° C. for 1 hr with 10 μl of the working solution and subsequently washed twice with PBS. After labeling with FLICA, 1% propidium iodide (PI) in PBS was used to assess dead cells. All samples were analyzed directly by flow cytometry to detect caspase activity (Bedner et al., 2000; Darzynkiewicz et al., 2011).

qRT-PCR from Mouse Sperm Lysates

Sperm pellets previously snap-frozen in PBS were thawed. RNA was extracted using the trizol method (Rio et al., 2010). The quality and quantity of the RNA were measured with NanoDrop 2000 from Thermo Scientific. RNA (2 μg) from each sample was taken for reverse transcription with dTVN and N9 primers and SuperScript III reverse transcriptase, all procured from Invitrogen. The resulting cDNA was used to perform real-time qPCR with SYBR Green on an Applied Biosystems 7900HT Real-Time PCR machine was used. To determine the relative expression of various DNA repair genes in mouse sperm, we used the ΔΔCt method, which makes use of the expression of housekeeping genes. GAPDH was used as housekeeping gene. Primers were procured from Integrated DNA technologies (IDT) (Table 3).

TABLE 2
Primers used for genotyping BRCA-mutant mice.

| | |
|---|---|
| B004 | CTG GGT AGT TTG TAA GCA TGC (SEQ ID NO: 1) |
| B005 | CAA TAA ACT GCT GGT CTC AGG (SEQ ID NO: 2) |
| B007 | ATC GCC TTC TAT CGC CTT CTT GAC GAG TTC |
| | (SEQ ID NO: 3) |

TABLE 3
mouse (M)-pecifie primers used for
sperm qRT-PCR.

| | |
|---|---|
| BRCA1 f-M | AGTTGTGATCGTC3-CAGCCAAGC |
| | (SEQ ID NO: 4) |
| BRCA1 r-M | ACAAGACGTGCCTTGCACAGCT |
| | (SEQ ID NO: 5) |
| Rad50 f-M | TGATAAGTTGTCTTGGGGTTTCC |
| | (SEQ ID NO: 6) |
| Rad50 r-M | CTGTGTCTGACGCACCTGT |
| | (SEQ ID NO: 7) |
| Rad51 f-M | GGCTGGTTTACTGTGTCTTT |
| | (SEQ ID NO: 8) |
| Rad51 r-M | TGGAAATACTCCTGATGCAG |
| | (SEQ ID NO: 9) |
| ATM f-M | TGGGTGGACAGGTGAACTTGCT |
| | (SEQ ID NO: 10) |
| ATM r-M | ACCCAAGCTTTCCATCCTGGGA |
| | (SEQ ID NO: 11) |
| Mre11 f-M | TCTGGCTCACGCTTCCACAACA |
| | (SEQ ID NO: 12) |
| Mre11 r-M | TTTTCAGGCATCTCGGGGCTGT |
| | (SEQ ID NO: 13) |
| DMC1 f-M | AGAAACATGGAATTAACGTGGCT |
| | (SEQ ID NO: 14) |
| DMC1 r-M | AAATGCAGTCAAGAATCCTGGTT |
| | (SEQ ID NO: 15) |
| GAPDH f-M | CTGGAGAAACCTGCCAAGTA (SEQ |
| | ID NO: 16) |
| GAPDH r-M | TGTTGCTGTAGCCGTATTCA (SEQ |
| | ID NO: 17) |

Embryo Collection & Quality Assessment BRCA1 WT female mice (n=7) were superovulated with intraperitioneal administration of 5 IU PMSG (pregnant mare's serum gonadotropin, Sigma-Aldrich) followed 72 h later with 5 IU human chorionic gonadotropin (Calbiochem, San Diego, Calif.). Mice were cohoused (2 female per male mouse) with either a BRCA1-mutant or WT male. The morning after mating, also known as embryonic day 0.5 (E0.5), female mice were checked for the presence of a vaginal plug as an indicator of mating. Those females with confirmed plugs were separated. On day 5, embryos were flushed from the uterus and observed for morphology. Implantation rate was also assessed using 1% Evans blue dye.

### Neutral Comet Assay

Slides were prepared 48 hours prior to experimentation by coating normal glass microscope slides with 500 μl normal melting point agar (NMPA, 0.5% w/v in PBS) solution and left to dry at room temperature for no less than 48 hours. Once samples were obtained, 10 μl of mouse sperm suspension was mixed with 90 μl of 0.5% low melting point agar (LMPA). Then, this 100 μl solution was added to the NMPA-coated slides. Coverslips were added and slides were placed on a metallic tray on ice and in the dark for 5 minutes. The cover slips were then removed and another layer of LMPA was added. Cover slips were subsequently replaced before the LMPA hardened. Slides were then kept in the dark on ice again for another 5 minutes. After ten minutes, the cover slips were removed and slides were placed in staining jars containing ice cold lysing solution (2.5 M NaCl, 100 mM EDTA, sodium N-laurylsacrosinate [SLS]. [1% w/v] and 10 mM Trizma base pH10, and 1% Triton-X-100 and 10% DMSO [immediately adding before use]) for 1 hour at 4° C. in a dark place.

After this lysis step, the slides were washed three times for 5 minutes with the cold electrophoresis buffer (0.089 M Tris base, 0.089 M boric acid and 2 mM EDTA [pH 8.3]), and then placed flatly in the electrophoresis tank face up for 30 minutes. This incubation period was used to achieve the unwinding of the DNA. Electrophoresis was then performed at 18 V for 60 minutes. After this, slides were washed twice in PBS for 5 minutes each. Before analysis could occur, the sperm within the slides needed to be fixed by two 10 minute washes in ethanol, and then air drying at room temperature. Ethidium bromide (50 μl at 10 μg/ml in water) was then added on the slide, and sealed with a cover slip. Slides were kept moist, wrapped in aluminum foil to prevent light exposure, and stored at 4° C. until analysis (Smart et al., 2008).

### Alkaline Comet Assay

Slides were prepared 48 hours prior to experimentation by coating normal glass microscope slides with 500 μl normal melting point agar (0.5% w/v in PBS) solution and left to dry at room temperature for no less than 48 hours. Then, 10 μl of mouse sperm suspension was mixed with 90 μl of 0.5% LMPA. Then this 100 μl solution was added to the NMPA-coated slides. Coverslips were added and slides were placed on a metallic tray on ice and in the dark for 5 minutes. The cover slips were removed and a 90 μl layer of LMPA was added. Cover slips were subsequently replaced before the LMPA hardened. Slides were then kept in the dark on ice again for another 5 minutes. After ten minutes, the cover slips were removed and slides were placed in staining jars containing ice cold lysing solution (2.5 M NaCl, 200 mM NaOH, 100 mM EDTA, 10 mM Trizma Base, 1% Triton X-100 and 10% DMSO [pH 10]) for 1.5 hours at 4° C. in a dark place.

After this lysis step, the slides were washed three times for 5 minutes with TE buffer (pH 8.0), and then placed flatly in the electrophoresis tank face up for 30 minutes to achieve the unwinding of the DNA. The alkaline electrophoresis buffer in this tank included 300 mM NaOH and 1 mM EDTA [pH13]. This incubation period was used to achieve the alkaline unwinding of the DNA. Electrophoresis was then performed at 25 V, 300 mA for 30 minutes. After this, slides were washed twice in neutralization buffer (0.4 M Tris, pH 7.5) for 5 minutes each. Before analysis could occur, the sperm within the slides needed to be fixed by draining the slides well on paper towels and then subjecting them to two 10 minute washes in ethanol, and then air drying at room temperature for 5 minutes. Ethidium bromide (50 μl at 10 μg/ml in water) was then added on the slide, and sealed with a cover slip. Slides were kept moist, wrapped in aluminum foil to prevent light exposure, and stored at 4° C. until analysis (Williams et al., 2014).

### Comet Assay Analysis

Slides from both alkaline and neutral comets were imaged by fluorescence microscopy using a Nikon OPTIPHOT microscope. ImageJ was used for comet assay analysis, which calculated tail length, tail moment, and % of DNA in the tail. Each image used had a dark background and light comets. The comets were “on scale” for the calculations to be correct (the brightest pixels of interest were below 255 on an 8-bit scale). Camera noise subtraction, flat field correction, and a background subtraction on the images was conducted before running the comet assay plugin for ImageJ. To analyze the slides, an oval around the sperm head was drawn and calculated and then an oval around the sperm tail was drawn and calculated. Each comet had two lines of results: the first line was the head values and the values were as follows: X and Y were coordinates of the Centroid, XM and YM were coordinates of the Center of Mass, IntDen and RawIntDen were the same and were the integrated density, TailLen was the tail length, TailMoment was the tail moment and % TailDNA was percent of total DNA that was in the tail. The “Center of Mass” is the brightness-weighted average of X Y coordinates of a selection. Center of Mass of the tail and Centroid of the head were used in calculating the tail length. The tail length as used here was the distance from the Centroid of the head to the Center of Mass of the tail. It was calculated as the Pythagorean distance between two points. The tail length variable is called CometLen in the code. Tail Moment was the length of the tail times the integrated density of the tail. Percent of DNA in the tail was the integrated density of the tail divided by the integrated density of the tail plus the integrated density of the head times 100. The % Tail DNA was used to compare the samples of each group (Russ, 1995).

### REFERENCES

1. Anbari K K, Ierardi-Curto L A, Silber J S, Asada N, Spinner N, Zackai E H, Belasco J, Morrissette J D, Dormans J P (2000). “Two primary osteosarcomas in a patient with Rothmund-Thomson syndrome”. Clin. Orthop. Relat. Res. 378: 213-23.
2. Alter B P (2003). “Cancer in Fanconi anemia, 1927-2001”. Cancer 97 (2): 425-40.
3. Aoki, M., Aoki, H., Ramanathan, R., Hait, N. C., and Takabe, K. (2016). Sphingosine-1-Phosphate Signaling in Immune Cells and Inflammation: Roles and Therapeutic Potential. Mediators of inflammation 2016, 8606878.
4. Bartkova J, Tommiska J, Oplustilova L, Aaltonen K, Tamminen A, Heikkinen T, Mistrik M. Aittomäki K, Blomqvist C, Heikkilä P, Lukas J. Nevanlinna H, Bartek J (2008). “Aberrations of the MRE11-RAD50-NBS1 DNA damage sensor complex in human breast cancer: MRE11 as a candidate familial cancer-predisposing gene”. Mol Oncol 2 (4): 296-316.
5. Battaglia et al., (1996). “Influence of maternal age on Meiotic spindle assembly in oocytes from naturally cycling women”. Human Reproduction 11: 2217-2222
6. Beckham, T. H., Cheng, J. C., Marrison, S. T., Norris, J. S., and Liu, X. (2013). Interdiction of sphingolipid metabolism to improve standard cancer therapies. Advances in cancer research 117, 1-36.
7. Bejarano, I., Monllor, F., Marchena, A. M., Ortiz, A., Lozano, G., Jimenez, M. I., Gaspar, P., Garcia, J. F., Pariente, J. A., Rodriguez, A. B., et al. (2014). Exogenous melatonin supplementation prevents oxidative stress-evoked DNA damage in human spermatozoa. J Pineal Res 57, 333-339.
8. Chandra et al., (2013) “Infertility and Impaired Fecundity in The United States, 1982-2010: Data From the Nationals Survey of Family Growth”. National Health Statistics Reports 67: 1-19
9. Chianese, C., Brilli, S., and Krausz, C. (2014). Genomic changes in spermatozoa of the aging male. Advances in experimental medicine and biology 791, 13-26.
10. Chrzanowska K H, Gregorek H, Dembowska-Bagińiska B, Kalina M A, Digweed M (2012). “Nijmegen breakage syndrome (NBS)”. Orphanet J Rare Dis 7: 13.
11. Fisch, H. (2009). “Older men are having children, but the reality of a male biological clock makes this trend worrisome”. Geriatrics 64, 14-17.
12. German J (1969). “Bloom's syndrome. I. Genetical and clinical observations in the first twenty-seven patients”. Am. J. Hum. Genet. 21 (2): 196-227.
13. Goriely et al., (2012) “Maternal Age Effect Mutations and Selfish Spermatogonial Selection: Causes and Consequences for Human Disease”. Am J Hum Genet. 90(2): 175-200
14. Guo, L., Ou. X., Li, H., and Han, Z. (2014). Roles of sphingosine-1-phosphate in reproduction. Reprod Sci 21, 550-554.
15. Hannun, Y. A. (1996). Functions of ceramide in coordinating cellular responses to stress. Science 274, 1855-1859.
16. Harris, I. D., Fronczak, C., Roth, L., and Meacham, R. B. (2011). Fertility and the Aging Male. Reviews in Urology 13, e184-e190.
17. Ishii, M., Egen, J. G., Klauschen, F., Meier-Schellersheim, M., Saeki, Y., Vacher, J., Proia, R. L., and Germain, R. N. (2009). Sphingosine-1-phosphate mobilizes osteoclast precursors and regulates bone homeostasis. Nature 458, 524-528.
18. Keimling M, Volcic M, Csernok A, Wieland B, Dörk T, Wiesmüller L (2011). “Functional characterization connects individual patient mutations in ataxia telangiectasia mutated (ATM) with dysfunction of specific DNA double-strand break-repair signaling pathways”. FASEB J. 25 (11): 3849-60.
19. Kong et al., (2012) “Rate of De Novo Mutations and the Importance of Father's Age to Disease Risk”. Nature 488 (7412): 471-5
20. Kuhnert, B., and Nieschlag, E. (2004). Reproductive functions of the ageing male. Hum Reprod Update 10, 327-339.
21. Lancaster J M, Powell C B, Chen L M, Richardson D L (2015). “Society of Gynecologic Oncology statement on risk assessment for inherited gynecologic cancer predispositions”. Gynecol. Oncol. 136 (1): 3-7.
22. Li, F., Turan, V., Lierman, S., Cuvelier, C., De Sutter. P., and Oktay, K. (2014). Sphingosine-1-phosphate prevents chemotherapy-induced human primordial follicle death. Hum Reprod 29, 107-113.
23. Markkanen E, Dorn J, Hübscher U (2013). “MUTYH DNA glycosylase: the rationale for removing undamaged bases from the DNA”. Front Genet 4: 18.
24. Meyer L A, Broaddus R R, Lu K H (2009). Library/Main Nav/Research and Clinical Trials/Cancer Control Journal/v16n1/14.pdf “Endometrial cancer and Lynch syndrome: clinical and pathologic considerations” Cancer Control 16 (1): 14-22.
25. Morita, Y., Perez, G. I., Paris, F., Miranda, S. R., Ehleiter, D., Haimovitz-Friedman. A., Fuks, Z., Xie, Z., Reed, J. C., Schuchman, E. H., et al. (2000). Oocyte apoptosis is suppressed by disruption of the acid sphingomyelinase gene or by sphingosine-1-phosphate therapy. Nat Med 6, 1109-1114.
26. Nagaraju G, Scully R (2007). “Minding the gap: the underground functions of BRCA1 and BRCA2 at stalled replication forks”. DNA Repair (Amst.) 6 (7): 1018-31.
27. Nicolai et al., (2015) “DNA Repair and Aging: the Impact of the p53 Family” Aging 7(12): 1050-1065
28. Nimonkar A V, Ozsoy A Z, Genschel J, Modrich P, Kowalczykowski S C (2008). “Human exonuclease 1 and BLM helicase interact to resect DNA and initiate DNA repair”. Proc. Natl. Acad. Sci. U.S.A. 105 (44): 16906-11.
29. Otala, M., Suomalainen, L., Pentikainen, M. O., Kovanen, P., Tenhunen, M., Erkkila, K., Toppari, J., and Dunkel, L. (2004). Protection from radiation-induced male germ cell loss by sphingosine-1-phosphate. Biol Reprod 70, 759-767.
30. Rapp A, Greulich K O (2004). “After double-strand break induction by UV-A, homologous recombination and nonhomologous end joining cooperate at the same DSB if both systems are available”. J. Cell. Sci. 117 (Pt 21): 4935-45.
31. Suomalainen. L., Hakala, J. K., Pentikainen, V., Otala, M., Erkkila, K., Pentikainen, M. O., and Dunkel, L. (2003). Sphingosine-1-phosphate in inhibition of male germ cell apoptosis in the human testis. The Journal of clinical endocrinology and metabolism 88, 5572-5579.
32. Singh D K, Ahn B, Bohr V A (2009). “Roles of RECQ helicases in recombination based DNA repair, genomic stability and aging”. Biogerontology 10 (3): 235-52.
33. Thompson L H, Hinz J M (2009). “Cellular and molecular consequences of defective Fanconi anemia proteins in replication-coupled DNA repair: mechanistic insights”. Mutat. Res. 668 (1-2): 54-72. doi:10.1016/j.mrfmmm.2009.02.00
34. Thompson L H, Schild D (2002). “Recombinational DNA repair and human disease”. Mutat. Res. 509 (1-2): 49-78.
35. Titus et al., (2013) “Impariment of BRCA1-related DNA double-strand Break Repair Leads to Ovarian Aging in Mice and Humans”. Sci Transl Med. 5(172): 172ra21
36. Tilly. J. L., and Kolesnick, R. N. (2007). Protection of the female reproductive system from natural and artificial insults (U.S. Pat. No. 7,195,775).
37. Winkle et al., (2009) “The correlation between male age, sperm quality and sperm DNA fragmentation in 320 men attending a fertility center” J. Assist Reprod Genet. 26(1): 41-46
38. Hobson et al. (2015) “Discovery of A-971432, An Orally Bioavailable Selective Sphingosine-1-Phosphate Receptor 5 (S1P5) Agonist for the Potential Treatment of Neurodegenerative Disorders”. J Med Chem. 2015 Dec. 10; 58(23):9154-70, FTY720 and SEW2871
39. Soleimani et al. (2011) “Enhancement of neoangiogenesis and follicle survival by sphingosine-1-phosphate in human ovarian tissue xenotransplants”. PLoS One 29; 6(4):e 19475.
40. Ayzenberg et al., 2016 Ther Clin Risk Manag 12: 261-272

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, and publications are cited throughout this application, the disclosures of which, particularly, including all disclosed chemical structures, are incorporated herein by reference. Citation of the above publications or documents is not intended as an admission that any of the foregoing is pertinent prior art, nor does it constitute any admission as to the contents or date of these publications or documents. All references cited herein are incorporated by reference to the same extent as if each individual publication, patent application, or patent, was specifically and individually indicated to be incorporated by reference.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A composition comprising live sperm, and at least one stabilizing agent effective in increasing the lifespan or stability of the sperm, and optionally a physiologically acceptable carrier.

2. The composition of claim 1, wherein the stabilizing agent comprises an effective amount sphingosine 1 phosphate (S1P), a pharmaceutically acceptable salt thereof, or an analog thereof.

3. The composition of claim 1, further comprising at least one cryoprotectant.

4. A kit comprising the composition of claim 2 and optionally a cryoprotectant.

5. The kit of claim 4 further comprising a device for self-administration.

6. A method of treating male reproductive system by administering a composition comprising sphingosine-1-phosphate, a pharmaceutically acceptable salt thereof, or an analog thereof, in an amount sufficient to enhance DNA repair to protect against a natural or artificial insult, wherein the administration is in vivo or ex vivo.

7. The method of claim 6 wherein the natural insult is aging and the artificial insult is chemotherapeutic drugs or radiation treatment.

8. A method to protect a male or a female subject against overall aging, reverse the effects of aging and to extend lifespan by administering to the subject a composition comprising sphingosine-1-phosphate, a pharmaceutically acceptable salt thereof, or an analog thereof in an effective amount sufficient to enhance DNA repair, wherein the administration is in vivo or ex vivo.

9. A method for improving functionality and/or fertility of sperm, comprising contacting the sperm with an effective amount of sphingosine 1 phosphate (S1P), a pharmaceutically acceptable salt thereof, or an analog thereof.

10. The method of claim 2, 6, 8 or 9 wherein the S1P analog is N,N-dimethylsphingosine-1-phosphate; N,N,N-trimethylsphingosine-1-phosphate; N-acetylsphingosine-1-phosphate; N-acylsphingosine-1-phosphate; sphingosine-1,3-diphosphate; sphingosine-3-phosphate; sphingosine-1-thiophosphate; N,N-dimethylsphingosine-1-thiophosphate; N,N,N-trimethylsphingosine-1-thiophosphate; or pharmaceutically acceptable salts thereof.

11. The method of claim 2, 8 or 9 wherein the effective amount is 0.001 milligram to 200 milligrams of S1P, a pharmaceutical acceptable salt thereof, or an analog thereof per kilogram body weight.

12. A method for improving an outcome in artificial insemination comprising: contacting an oocyte with an effective amount of the composition of claim 1.

13. The method of claim 12 wherein the artificial insemination includes intrauterine, intracervical, or intravaginal routes, and assisted reproductive techniques include gamete intrafallopian transfer (“GIFT”), zygote intrafallopian transfer (“ZIFT”), intracytoplasmic sperm injection (“ICSI”), and in vitro fertilization followed by embryo cryopreservation or transfer.

14. The method of claim 12 further comprising the steps of:
(i) retrieving an oocyte via a transvaginal oocyte retrieval with or without prior controlled ovarian stimulation;
(ii) retrieving sperms via masturbation, ejaculation, electro-ejaculation, prostate massage, centrifugation of urine post ejaculation in case of retrograde ejaculation, testicular sperm extraction (TESE), testicular sperm aspiration (TESA), microscopic testicular sperm extraction (micro-TESE), from fresh testicular tissues, frozen testicular tissues, freshly removed testis or frozen testis;
(iii) treating the sperms, testis or testicular tissues with S1P, a pharmaceutically acceptable salt thereof or an analog thereof from 1 min to 3 days in vitro prior to artificial insemination, in vitro fertilization, intracytoplasmic sperm injection, gamete intrafallopian transfer (“GIFT”) or zygote intrafallopian transfer (“ZIFT”);
(iv) combining the sperms and oocyte in the presence of S1P to form an embryo; and
(v) transferring the embryo into the fallopian tube or uterine cavity of a subject via laparoscope, hysteroscope, or optionally, cryopreserving the embryo.

15. A method for cryopreserving sperm comprising: (a) contacting the composition of claim 1 with a cryoprotectant; and (b) storing the composition at a temperature of about 4° C. to about −196° C.

16. A method of protecting the reproductive system of a male subject against a chemical or radiation insult, comprising: administering in vivo or ex vivo to said male subject a composition comprising an agent that antagonizes one or more acid sphingomyelinase (ASMase) gene products and/or enhances DNA repair, wherein said agent is a lysophospholipid, in an amount sufficient to protect the reproductive system of said male subject from pre-mature aging or destruction caused by said chemical or radiation insult.

17. The method of claim 16, wherein said chemical insult includes cytotoxic factors, chemotherapeutic drugs, hormone deprivation, growth factor deprivation, cytokine deprivation, cell receptor antibodies, or a combination thereof.

18. The method of claim 16, wherein said chemotherapeutic drug includes cyclophosphamide, melphalan, any other member of the alkylating agents category of chemotherapeutics, doxorubicin or any other member of the topoisomerase inhibitor category of chemotherapeutic drugs, 5-fluorouracil (5FU), methotrexate, or any other member of the antimetabolite category chemotherapeutic drugs, vinblastine, actinomycin D, etoposide, cisplatin, taxotere, taxol or a combination thereof.

19. The method of claim 16, wherein said radiation insult includes ionization radiation, x-ray, infrared radiation, ultrasound radiation, heat, or a combination thereof.

20. The method of claim 16, wherein said radiation insult includes an invasive radiation therapy, a non-invasive radiation therapy, or a combination thereof.

21. The method of claim 16, wherein said male reproductive system comprises testes.

22. The method of claim 16, wherein said male reproductive system comprises sperms.

23. The method of claim 16, wherein said male is in a reproductive age.

24. The method of claim 16, wherein said male is in a pre-reproductive age.

25. The method of claim 16, wherein said male is in a post-reproductive age.

26. The method of claim 16, wherein said lysophospholipid is a sphingolipid compound, pharmaceutical salt thereof or an analog thereof.

27. The method of claim 16, wherein said sphingolipid compound is S1P, a pharmaceutical salt thereof or an analog thereof.

28. The method of claim 16, wherein said composition is administered at least once from about fifteen days to about two days prior to exposure to said insult.

29. The method of claim 16, wherein said composition is administered at about seven days to about two hours prior to exposure to said insult.

30. The method of claim 16, wherein said composition is administered regularly for a continuous period of time.

31. The method of claim 16, wherein said composition is administered orally, intravascularly, intraperitoneally, subcutaneously, intramuscularly, inter-uterine, intra-ovarian, intratesticular, inta-urethral, rectally, topically, or a combination thereof.

32. The method of claim 16, wherein said chemical or radiation insult is a result of a therapy against a disease or a disorder.

33. The method of claim 32, wherein said disease or disorder comprises cancer, rheumatoid arthritis, angioplasy, or restenosis.

34. The method of claim 33, wherein said cancer comprises colon carcinoma, pancreatic cancer, breast cancer, ovarian cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acute lymphocytic leukemia and acute myelocytic leukemia, chronic leukemia and polycythemia vera, lymphoma (Hodgkin's disease and non-Hodgkin's disease), multiple myeloma, Waldenstrom's macroglobulinemia, or a combination thereof.

35. The method of claim 16, wherein said administration is an ex vivo administration.

36. The method of claim 16, wherein said administration is an in vivo administration.

37. The method of claim 16, wherein said male subject is exposed to a chemical insult prior to said radiation insult.

38. The method of claim 16, wherein said male subject is exposed to a radiation insult prior to said chemical insult.

39. The method of claim 38, wherein administration of said composition is terminated prior to, concurrently with or subsequent to said chemical insult.

40. The method of claim 37, wherein administration of said composition is terminated prior to, concurrently with or subsequent to said radiation insult.

41. A method to enhance DNA repair or reduce cancer risk of a subject comprising administering an effective amount of acid sphingomyelinase (ASMase) antagonist.

42. The method of claim 41 wherein the antagonist is lysophospholipid.

43. The method of claim 42, wherein said lysophospholipid is a sphingolipid compound, pharmaceutical salt thereof or an analog thereof.

44. The method of claim 43, wherein said sphingolipid compound is S1P, a pharmaceutical salt thereof or an analog thereof.
